SEQUENCE LISTING (71 sequences)

SEQ ID NO: 1 - protein, 25 aa, Artificial Sequence
  Description of Artificial Sequence Synthetic peptide
MKKLAFAITAASGAAAVLSHHDAEA

SEQ ID NO: 2 - protein, 30 aa, Artificial Sequence
  Description of Artificial Sequence Synthetic polypeptide
WLDNRAFSKKFVPVVMATSVALFFLNLAFA

SEQ ID NO: 3 - protein, 29 aa, Artificial Sequence
  Description of Artificial Sequence Synthetic peptide
MAKKFNYKLPSMVALTLFGTAFTAHQANA

SEQ ID NO: 4 - protein, 27 aa, Artificial Sequence
  Description of Artificial Sequence Synthetic peptide
MKKRFLSICTMTIAALATTTMVNTSYA

SEQ ID NO: 5 - protein, 30 aa, Artificial Sequence
  Description of Artificial Sequence Synthetic polypeptide
NLKKQSKLILIFICIFTFFIMIIQSQFLMG

SEQ ID NO: 6 - protein, 26 aa, Artificial Sequence
  Description of Artificial Sequence Synthetic peptide
MKIFKLTSLTLAALTLAFPFSHVAQA

SEQ ID NO: 7 - protein, 28 aa, Artificial Sequence
  Description of Artificial Sequence Synthetic peptide
MKKTVIASTLAVSLGIAGYGLSGHEAHA

SEQ ID NO: 8 - protein, 27 aa, Artificial Sequence
  Description of Artificial Sequence Synthetic peptide
MKKNKFLVYLLSTALITPTFATQTAFA

SEQ ID NO: 9 - protein, 35 aa, Artificial Sequence
  Description of Artificial Sequence Synthetic polypeptide
MKTRQNKYSIRKFSVGASSILIAALLFMGGGSAQA

SEQ ID NO: 10 - protein, 37 aa, Artificial Sequence
  Description of Artificial Sequence Synthetic polypeptide
MKNNNETRRFSIRKYTVGVVSIITGITIFVSGQHAQA

SEQ ID NO: 11 - protein, 30 aa, Artificial Sequence
  Description of Artificial Sequence Synthetic polypeptide
MKKKLSYMITIMLAFTLSLALGLFFNSAHA

SEQ ID NO: 12 - protein, 27 aa, Artificial Sequence
  Description of Artificial Sequence Synthetic peptide
MKKRFLSICTMTIAALATTTMVNTSYA

SEQ ID NO: 13 - protein, 13 aa, Artificial Sequence
  Description of Artificial Sequence Synthetic peptide
GRKKRRQRRRPPQ

SEQ ID NO: 14 - protein, 27 aa, Artificial Sequence
  Description of Artificial Sequence Synthetic peptide
GWTLNSAGYLLGKINLKALAALAKKIL

SEQ ID NO: 15 - protein, 18 aa, Artificial Sequence
  Description of Artificial Sequence Synthetic peptide
KLALKLALKALKAALKLA

SEQ ID NO: 16 - protein, 30 aa, Artificial Sequence
  Description of Artificial Sequence Synthetic polypeptide
WEAKLAKALAKALAKHLAKALAKALKACEA

SEQ ID NO: 17 - protein, 21 aa, Artificial Sequence
  Description of Artificial Sequence Synthetic peptide
KETWWETWWTEWSQPKKKRKV

SEQ ID NO: 18 - protein, 9 aa, Artificial Sequence
  Description of Artificial Sequence Synthetic peptide
RRRRRRRRR

SEQ ID NO: 19 - protein, 24 aa, Artificial Sequence
  Description of Artificial Sequence Synthetic peptide
LGTYTQDFNKFHTFPQTAIGVGAP

SEQ ID NO: 20 - protein, 11 aa, Artificial Sequence
  Description of Artificial Sequence Synthetic peptide
ACHSALTKHCG

SEQ ID NO: 21 - protein, 11 aa, Artificial Sequence
  Description of Artificial Sequence Synthetic peptide
ACKTGSHNQCG

SEQ ID NO: 22 - protein, 11 aa, Artificial Sequence
  Description of Artificial Sequence Synthetic peptide
ACTGSTQHQCG

SEQ ID NO: 23 - protein, 11 aa, Artificial Sequence
  Description of Artificial Sequence Synthetic peptide
ACTHGQTQSCG

SEQ ID NO: 24 - protein, 11 aa, Artificial Sequence
  Description of Artificial Sequence Synthetic peptide
ACSATLQHSCG

SEQ ID NO: 25 - protein, 11 aa, Artificial Sequence
  Description of Artificial Sequence Synthetic peptide
ACSLTVNWNCG

SEQ ID NO: 26 - protein, 11 aa, Artificial Sequence
  Description of Artificial Sequence Synthetic peptide
ACTSTGRNACG

SEQ ID NO: 27 - protein, 11 aa, Artificial Sequence
  Description of Artificial Sequence Synthetic peptide
ACSASTNHNCG

SEQ ID NO: 28 - protein, 15 aa, Artificial Sequence
  Description of Artificial Sequence Synthetic peptide
RRWRRWNRFNRRRCR

SEQ ID NO: 29 - protein, 29 aa, Artificial Sequence
  Description of Artificial Sequence Synthetic peptide
KLAKLAKKLAKLAKRRWRRWNRFNRRRCR

SEQ ID NO: 30 - protein, 6 aa, Artificial Sequence
  Description of Artificial Sequence Synthetic peptide
LVGVFH

SEQ ID NO: 31 - protein, 7 aa, Artificial Sequence
  Description of Artificial Sequence Synthetic peptide
DRTTLTN

SEQ ID NO: 32 - protein, 11 aa, Artificial Sequence
  Description of Artificial Sequence Synthetic peptide
ACSSSPSKHCG

SEQ ID NO: 33 - protein, 12 aa, Artificial Sequence
  Description of Artificial Sequence Synthetic peptide
HIITDPNMAEYL

SEQ ID NO: 34 - protein, 36 aa, Artificial Sequence
  Description of Artificial Sequence Synthetic polypeptide
GPSQPTYPGDDAPVRDLIRFYRDLRRYLNVVTRHRY

SEQ ID NO: 35 - protein, 26 aa, Artificial Sequence
  Description of Artificial Sequence Synthetic peptide
WTLNSAGYLLGKINLKALAALAKKIL

SEQ ID NO: 36 - protein, 11 aa, Artificial Sequence
  Description of Artificial Sequence Synthetic peptide
YARAAARQARA

SEQ ID NO: 37 - protein, 18 aa, Artificial Sequence
  Description of Artificial Sequence Synthetic peptide
KWKLFKKIGAVLKVLTTG

SEQ ID NO: 38 - protein, 16 aa, Artificial Sequence
  Description of Artificial Sequence Synthetic peptide
RQIKIWFQNRRMKWKK

SEQ ID NO: 39 - protein, 11 aa, Artificial Sequence
  Description of Artificial Sequence Synthetic peptide
YGRKKRRQRRR

SEQ ID NO: 40 - protein, 23 aa, Artificial Sequence
  Description of Artificial Sequence Synthetic peptide
ALFLGWLGAAGSTMGAPKKKRKV

SEQ ID NO: 41 - protein, 7 aa, Artificial Sequence
  Description of Artificial Sequence Synthetic peptide
TGSTQHQ

SEQ ID NO: 42 - protein, 12 aa, Artificial Sequence
  Description of Artificial Sequence Synthetic peptide
ACNAHQARSTCG

SEQ ID NO: 43 - protein, 28 aa, Artificial Sequence
  Description of Artificial Sequence Synthetic peptide
WYSCNVCGKAFVLSRHLNRHLRVHRRAT

SEQ ID NO: 44 - protein, 9 aa, Artificial Sequence
  Description of Artificial Sequence Synthetic peptide
LEISSTCDA

SEQ ID NO: 45 - protein, 9 aa, Artificial Sequence
  Description of Artificial Sequence Synthetic peptide
LGISSTCNA

SEQ ID NO: 46 - protein, 9 aa, Artificial Sequence
  Description of Artificial Sequence Synthetic peptide
LQVDDIPSA

SEQ ID NO: 47 - protein, 9 aa, Artificial Sequence
  Description of Artificial Sequence Synthetic peptide
LKISSTCHA

SEQ ID NO: 48 - protein, 8 aa, Artificial Sequence
  Description of Artificial Sequence Synthetic peptide
ETANKSNE

SEQ ID NO: 49 - protein, 10 aa, Artificial Sequence
  Description of Artificial Sequence Synthetic peptide
ETANESNEGT

SEQ ID NO: 50 - protein, 10 aa, Artificial Sequence
  Description of Artificial Sequence Synthetic peptide
ETANASNEGT

SEQ ID NO: 51 - protein, 10 aa, Artificial Sequence
  Description of Artificial Sequence Synthetic peptide
ETANKSNAGT

SEQ ID NO: 52 - protein, 10 aa, Artificial Sequence
  Description of Artificial Sequence Synthetic peptide
ATANKSNEGT

SEQ ID NO: 53 - protein, 10 aa, Artificial Sequence
  Description of Artificial Sequence Synthetic peptide
ETANKSNKGT

SEQ ID NO: 54 - protein, 39 aa, Artificial Sequence
  Description of Artificial Sequence Synthetic polypeptide
KTDTESHNHSSLGTENKNVLDINSSSHNIKPSQNKSYPS

SEQ ID NO: 55 - protein, 10 aa, Artificial Sequence
  Description of Artificial Sequence Synthetic peptide
DTNSDIAKQD

SEQ ID NO: 56 - protein, 10 aa, Artificial Sequence
  Description of Artificial Sequence Synthetic peptide
DTTTDIAKQE

SEQ ID NO: 57 - protein, 10 aa, Artificial Sequence
  Description of Artificial Sequence Synthetic peptide
DTSADIANQE

SEQ ID NO: 58 - protein, 54 aa, Artificial Sequence
  Description of Artificial Sequence Synthetic polypeptide
GDGEEKGSYAETHGLTADDVESINALNERALTLGQPGKPPKELPPSASAPSRAP

SEQ ID NO: 59 - protein, 58 aa, Artificial Sequence
  Description of Artificial Sequence Synthetic polypeptide
ASGGDEEWEGSYAATHGLTAEDVKNINALNKRALTAGQPGNFPAELPPSATALFRAPD

SEQ ID NO: 60 - protein, 53 aa, Artificial Sequence
  Description of Artificial Sequence Synthetic polypeptide
ADSGDGAKKGSYAETHGLTAHDVKNINALNERAPALGQPGKPPAGAPPFRTPA

SEQ ID NO: 61 - protein, 58 aa, Artificial Sequence
  Description of Artificial Sequence Synthetic polypeptide
ASGDDEEREGSYAETHGLTAEDVKNINALNKRALTAGQPGNSLTELPPSVSALFRAPD

SEQ ID NO: 62 - protein, 45 aa, Artificial Sequence
  Description of Artificial Sequence Synthetic polypeptide
DNGAGEETKSYAETYRLTADDVANINALNESAPAASSAGPSFRAP

SEQ ID NO: 63 - protein, 53 aa, Artificial Sequence
  Description of Artificial Sequence Synthetic polypeptide
ADAGDGAKEGSYAETHGLTAHDVKNINALNERALAVGQPGKPPAGAPPFRTPA

SEQ ID NO: 64 - protein, 58 aa, Artificial Sequence
  Description of Artificial Sequence Synthetic polypeptide
ASRGDGEWEGSYAETHGLTAEDVKNINALNKRALTAGQPGNSAAELPPSASALFRAPD

SEQ ID NO: 65 - DNA, 279 bp, Artificial Sequence
  Description of Artificial Sequence Synthetic polynucleotide
atgttttcactctttttttatttatttgagcgctaattgttatttatcatttgatagtctcaaaaattttattaatgagtaagttactaataattgtgtccaaattcttataaaaaatattttaaaaaataaatataatatttaatataatgtaaacagatagggtttttattttaacatttaggcatttaatcacatattttatcagtttatatatataatttaattgtaacagctgttattaaaaaaataaattttataatttttggaggttatcat

SEQ ID NO: 66 - protein, 6 aa, Artificial Sequence
  Description of Artificial Sequence Synthetic peptide
ENYFQG

SEQ ID NO: 67 - protein, 4 aa, Artificial Sequence
  Description of Artificial Sequence Synthetic peptide
  MOD_RES (2)..(2): Glu or Asp
IXGR

SEQ ID NO: 68 - protein, 7 aa, Artificial Sequence
  Description of Artificial Sequence Synthetic peptide
LVPRGSH

SEQ ID NO: 69 - protein, 5 aa, Artificial Sequence
  Description of Artificial Sequence Synthetic peptide
  MOD_RES (1)..(4): Glu or Asp
XXXXK

SEQ ID NO: 70 - protein, 6 aa, Artificial Sequence
  Description of Artificial Sequence Synthetic peptide
PGAAHY

SEQ ID NO: 71 - protein, 7 aa, Artificial Sequence
  Description of Artificial Sequence Synthetic peptide
LEVLFQG

(19) United States
(12) Patent Application Publication
(10) Pub. No.: US 2021/0070823 A1
(43) Pub. Date: Mar. 11, 2021

(54) METHODS AND COMPOSITIONS FOR TREATING ATOPIC DERMATITIS WITH RECOMBINANT MICROORGANISMS

(71) Applicant: Azitra Inc, Farmington, CT (US)

(72) Inventor: Travis Michael Whitfill, Dallas, TX (US)

(21) Appl. No.: 16/839,995

(22) Filed: Apr. 3, 2020

Related U.S. Application Data
(60) Provisional application No. 62/829,693, filed on Apr. 5, 2019.

(51) Int. Cl. C07K 14/54; A61K 35/74; A61P 17/00

(52) U.S. Cl. CPC C07K 14/5406; A61K 38/00; A61P 17/00; A61K 35/74

(57) ABSTRACT

The present disclosure provides isolated plasmids, recombinant microorganisms, kits, and methods for the treatment of skin diseases, in particular atopic dermatitis.

### RELATED APPLICATIONS

This application claims priority to U.S. Provisional Application No. 62/829,693, filed on Apr. 5, 2019, the entire contents of which is incorporated by reference in its entirety herein.

### SEQUENCE LISTING

The instant application contains a Sequence Listing which has been submitted electronically in ASCII format and is hereby incorporated by reference in its entirety. Said ASCII copy, created on Mar. 31, 2020, is named 129062-00703_SL.txt and is 23,635 bytes in size.

### BACKGROUND

Atopic dermatitis (AD), or eczema, is a chronic, pruritic, inflammatory skin disease common in children but also prevalent in many adults. The primary features of the disease are dry, scaly, itchy skin. Notably, AD affects 5-20% of children worldwide,¹ and some form of AD affects 11% of the U.S. population,² or about 35 million people. Recent focus on the pathogenesis of AD has focused on impaired epidermal barrier function as a key driver of the disease.³ Current treatment options for AD include skin hydration, bleach baths, dietary interventions, antimicrobials, systemic immunomodulatory agents, and topical corticosteroids (TCS).⁴ However, despite numerous options, few provide long-lasting resolution of symptoms, AD recurrence is the norm for most individuals, and significant safety issues remain a concern with TCS or calcineurin inhibitors. Moreover, the 2013 National Health and Wellness Survey revealed significant associated burden on AD patients, who, compared with non-AD patients, reported higher uses of healthcare resources, lower health-related quality of life, and nearly twice as much lost work productivity.⁵ AD patients also had markedly higher prevalence of comorbidities such as allergies, asthma, and depression.⁵ Accordingly, there is a large unmet need in view of the significant burden atopic dermatitis has on the healthcare system.

Recent research has shed some light on the pathophysiology of AD and has revealed that, concurrent with a skin barrier defect, there is an activated inflammatory response, particularly driven by IL-4/IL-13. Dupilumab, marketed as DUPIXENT by Regeneron and Sanofi, is an IL-4R monoclonal antibody that has shown remarkable results in Phase III clinical trials with up to 39% of AD patients seeing complete clearing of the disease. However, this antibody must be injected, is very costly (S37,000 per year), and is approved for severe AD patients, which comprise a small segment of the total AD population. Concurrently, atopic dermatitis is often characterized by dysbiosis (or a microbial imbalance, the severity of which is associated with disease severity), which is a notable feature of atopic dermatitis, and a lack of diversity of the skin microbiome, which is dominated by Staphylococcus aureus during atopic dermatitis flares and untreated skin. Diverse communities of microorganisms populate the skin, and a square centimeter can contain up to a billion microorganisms.³⁸ These diverse communities of bacteria, fungi, mites and viruses can provide protection against disease and form dynamic, yet distinct niches on the skin.³⁹ Recent work suggests that skin commensal microorganisms are essential to maintaining healthy skin and maintaining the skin barrier. Increasing evidence has associated altered microbial communities or dysbiosis in the skin with cutaneous diseases^{38,40}. Dysbiosis is a driving feature of AD, with the cutaneous microbiome dominated by S. aureus during flares characterized by a lack of microbial diversity—the extent of which is associated with disease severity 17-21 Notably, it has been shown that IL-4 and IL-13 downregulate antimicrobial peptide (AMP) production in AD skin,^{22,23} which may contribute to AD-associated dysbiosis.

Engineered probiotics are a novel approach based on leveraging the skin microbiome for therapeutic purposes. Notably, an engineered probiotic has important advantages over other methods of drug delivery, as it will establish residence on the patient's skin and continuously and stably deliver therapeutic proteins in situ. Furthermore, certain strains of Staphylococcus epidermidis (SE) have demonstrated important beneficial immuno-modulatory and anti-pathogen effects in the skin, which are relevant to atopic dermatitis disease phenotype and severity.

The present disclosure has the surprising advantage of providing methods and compositions for treating skin diseases, e.g., AD, using a genetically engineered, recombinant strain of Staphylococcus epidermidis as a skin drug delivery system that secretes human a mutant IL-4 protein to address the pathophysiology of atopic dermatitis. The benefits of this disclosure include its safety as a non-steroidal treatment option, its efficacy due to the disclosure's combination of benefits from the secretion of IL-4 along with the benefits of the topical application of Staphylococcus epidermidis, and its ability to be therapeutically effective at even a low frequency of application (no more than once a day).

The present disclosure therefore addresses the need in the art for novel and effective targeted therapies for the treatment and/or prevention of AD.

### SUMMARY OF THE INVENTION

The disclosure relates to methods and compositions for treating inflammatory skin diseases comprising an engineered microorganism capable of expressing a mutant IL-4, which is an IL-4R/IL-13 antagonist. In particular, the disclosure relates to a recombinant strain of skin commensal Staphylococcus epidermidis (SE) that secretes a mutant IL-4, which is an IL-4R/IL-13 antagonist. These engineered probiotics of the present disclosure are a novel approach predicated on leveraging the skin microbiome for therapeutic purposes. Notably, the described engineered probiotics have important advantages over other methods of drug delivery, as it will establish residence on the patient's skin and continuously and stably deliver therapeutic proteins in situ. Furthermore, certain strains of SE have shown to have important beneficial immunomodulatory and anti-pathogen effects in the skin, which are relevant to remediating AD disease phenotype and severity.

In a first aspect, the disclosure provides a recombinant microorganism capable of secreting a polypeptide, wherein the recombinant microorganism comprises an expression vector comprising a first coding sequence comprising a gene capable of expressing the polypeptide; and a second coding sequence comprising a gene capable of expressing a cell penetrating peptide (CPP). In some embodiments, the CPP is at the C-terminus. In some embodiments, the CPP is at the N-terminus. In some embodiments, a CPP is at both the C- and N-termini. In some embodiments, the recombinant microorganism further comprises a third coding sequence comprising a gene capable of expressing an export or secretion or signal peptide. In some embodiments, the recombinant microorganism further comprises a fourth coding sequence comprising a pro-peptide. According to some embodiments, the pro-peptide is included to assist with the export and/or the folding of the exported protein. In some embodiments, the expression of the first coding sequence, second coding sequence and third coding sequence is under the control of one or more promoters. According to some embodiments, a promoter is an inducible promoter. According to some embodiments, a promoter is a constitutive promoter. According to some embodiments, a promoter is a dual promoter. In some embodiments, the expression of the first coding sequence, second coding sequence, the third coding sequence and the fourth coding sequence is under the control of a promoter. In another embodiment, the arrangement of the first coding sequence, second coding sequence and third coding sequence are in-frame. In another embodiment, the arrangement of the first coding sequence, second coding sequence, third coding sequence and fourth coding sequence are in-frame. In another embodiment, the first coding sequence, second coding sequence and third coding sequence are operably linked to one or more promoters. According to some embodiments, a promoter is an inducible promoter. According to some embodiments, a promoter is a constitutive promoter. In another embodiment, the first coding sequence, second coding sequence, third coding sequence and fourth coding sequence are operably linked to a promoter. In some embodiments, the recombinant microorganism is a bacterium, or a combination of bacteria. In another embodiment, the polypeptide is a mutant IL-4, or a variant thereof. In some embodiments, the microorganism secretes a mutant IL-4 protein. In another embodiment, the mutant IL-4 inhibits IL-13/IL-4 responses. In another embodiment, the mutant IL-4 is [R121D, Y124D]-IL-4. In some embodiments, the microorganism is selected from the group consisting of Bifidobacterium, Brevibacterium, Cutibacterium (formerly known as Propionibacterium), Lactococcus, Streptococcus, Staphylococcus, Lactobacillus, Enterococcus, Pediococcus, Leuconostoc, and Oenococcus, or combinations thereof. In a further embodiment, the recombinant microorganism is Staphylococcus epidermidis.

In another aspect, the disclosure features a method for producing a live biotherapeutic composition, the method comprising (a) transfecting a cell with (i) a first coding sequence comprising a nucleic acid sequence capable of expressing a therapeutic polypeptide, and (ii) a second coding sequence comprising a nucleic acid sequence capable of expressing a cell penetrating peptide; and (b) allowing the transfected cell to produce a therapeutic polypeptide fusion protein; and (c) obtaining the live biotherapeutic composition. In some embodiments, the method further comprises (iii) transfecting the cell with a third coding sequence comprising a nucleic acid sequence capable of expressing an export or secretion or signal peptide. In some embodiments, the method further comprises (iv) transfecting the cell with a fourth coding sequence comprising a nucleic acid sequence capable of expressing a pro-peptide. In another embodiment, the first coding sequence, second coding sequence and third coding sequence are arranged in a single plasmid or integrated in genome at the site that will be neutral or enhancing the expression. In another embodiment, the first coding sequence, second coding sequence, third coding sequence and fourth coding sequence are arranged in a single plasmid or integrated in genome at the site that will be neutral or enhancing the expression. In some embodiments, the arrangement of the first coding sequence, second coding sequence and third coding sequence are operably linked to a promoter or more than one linked promoter. In some embodiments, the arrangement of the first coding sequence, second coding sequence, third coding sequence and fourth coding sequence are operably linked to one or more promoters. According to some embodiments, a promoter is an inducible promoter. According to some embodiments, a promoter is a constitutive promoter. In some embodiments, the cell is selected from the group consisting of wherein the microorganism is selected from the group consisting of Bifidobacterium, Brevibacterium, Cutibacterium (formerly known as Propionibacterium), Lactococcus, Streptococcus, Staphylococcus, Lactobacillus, Enterococcus, Pediococcus, Leuconostoc, or Oenococcus, or combinations thereof. In a further embodiment, the cell is Staphylococcus epidermidis. In some embodiments, the therapeutic polypeptide is a mutant IL-4, or a variant thereof. In some embodiments, the mutant IL-4 inhibits IL-13/IL-4 responses. In some embodiments, the mutant IL-4 is [R121D, Y124D]-IL-4.

In another aspect, the disclosure features a composition obtained by the method described in the any of the aspects and embodiments herein. In some embodiments, the composition comprises a pharmaceutically acceptable carrier, wherein the pharmaceutically acceptable carrier is selected from the group consisting of an aqueous solution, an emulsion, a cream, a lotion, a gel, or an ointment.

In another aspect, the disclosure features a live biotherapeutic composition comprising a recombinant microorganism wherein the recombinant microorganism comprises (i) a first coding sequence comprising a nucleic acid sequence capable of expressing a therapeutic polypeptide; (ii) a second coding sequence comprising a nucleic acid sequence capable of expressing a cell penetrating peptide; (iii) a third coding sequence comprising a nucleic acid sequence capable of expressing an export or secretion or signal peptide; and (iv) a promoter(s) operably linked to the first coding sequence, the second coding sequence and the third coding sequence; wherein the first coding sequence, second coding sequence and third coding sequence are capable of expressing an IL-4 mutant polypeptide, or variant thereof. In another aspect, the disclosure features a live biotherapeutic composition comprising a recombinant microorganism wherein the recombinant microorganism comprises (i) a first coding sequence comprising a nucleic acid sequence capable of expressing a therapeutic polypeptide; (ii) a second coding sequence comprising a nucleic acid sequence capable of expressing a cell penetrating peptide; (iii) a third coding sequence comprising a nucleic acid sequence capable of expressing an export or secretion or signal peptide; (iv) a fourth coding sequence comprising a pro-peptide and (v) a promoter(s) operably linked to the first coding sequence, the second coding sequence, the third coding sequence and the fourth coding sequence; wherein the first coding sequence, second coding sequence, third coding sequence and fourth coding sequence are capable of expressing an IL-4 mutant polypeptide, or variant thereof. In some embodiments, the recombinant microorganism is Staphylococcus epidermidis. In some embodiments, the signal peptide exports the IL-4 mutant protein, or variant thereof, out of the recombinant microorganism. In some embodiments, the cell penetrating peptide facilitates the entry of the IL-4 mutant polypeptide, or variant thereof, into a human keratinocyte. In some embodiments, the pro-peptide helps with the export and folding of the exported protein. In some embodiments, the composition comprises a pharmaceutically acceptable carrier, wherein the pharmaceutically acceptable carrier is selected from the group consisting of an aqueous solution, an emulsion, a cream, a lotion, a gel, or an ointment. In some embodiments, the composition comprises an antioxidant. In some embodiments, the antioxidant is selected from ascorbic acid (Vitamin C), α-tocopherol (Vitamin E), β-tocopherol, γ-tocopherol, δ-tocopherol, ε-tocopherol, ζι-tocopherol, ζ2-tocopherol, η-tocopherol, and retinol (Vitamin A) as well as derivatives thereof, e.g., esters.

In another aspect, the disclosure features a kit comprising the composition of any one of the aspects and embodiments described herein, and instructions for use.

In another aspect, the disclosure features a method of treating an abnormal skin condition comprising administering to a subject in need thereof the composition of any one of the aspects and embodiments herein. In some embodiments, the cell culture composition is a living cell culture composition. In some embodiments, the skin condition is atopic dermatitis (AD). In some embodiments, the AD is moderate-to-severe AD. In some embodiments, the subject is resistant, non-responsive or inadequately responsive to treatment by either a topical corticosteroid (TCS) or a calcineurin inhibitor. In some embodiments, the subject exhibits an elevated level of at least one AD-associated biomarker prior to, or at the time of treatment. In some embodiments, administration of the cell culture composition to the subject results in an improvement in an AD-associated parameter, wherein the improvement in the AD-associated parameter is selected from the group consisting of (a) a decrease from baseline in Investigator's Global Assessment (IGA) score; (b) a decrease from baseline in Body Surface Area Involvement of Atopic Dermatitis (BSA) score; (c) a decrease from baseline in Eczema Area and Severity Index (EASI) score; (d) a decrease from baseline in SCORAD score; (e) a decrease from baseline in 5-D Pruritus Scale; and (f) a decrease from baseline in Pruritus Numeric Rating Scale (NRS) score.

In one aspect, the disclosure features a composition comprising a mutant IL-4, or a variant thereof. In some embodiments, the IL-4 mutant, or variant thereof, inhibits IL-13/IL-4 responses. In another embodiment, the IL-4 mutant, or variant thereof, is [R121D, Y124D]-IL-4. According to some embodiments of any of the aspects or embodiments herein, the IL-4 mutant, or variant thereof, is codon optimized.

### BRIEF DESCRIPTION OF THE DRAWINGS

FIG. 1 is a schematic that shows the design of MUT-IL-4 producing Staphylococcus epidermidis (SE). The live biotherapeutic product (LBP) is comprised of a gene consisting of a promoter (sarA), RBS, secretion or signal peptide (SecA), mutant IL-4, and a cell import signal peptide.

FIG. 2A to FIG. 21 shows an S. epidermidis based protein delivery system. FIG. 2A shows construct design comprised of a promoter, RBS (ribosome binding site), export signal. FIG. 2B is a graph that shows colonization GFP secreting S. epidermidis onto mouse skin tails. n=3. 10e9 CFU were applied to 1 cm2 of skin, and skin was recultured to determined CFU count over 7 days. FIG. 2C is a graph that shows characterization of a selection of promoters for tunable control of protein expression (GFP used as reporter). FIG. 2D is a graph that shows characterization of export signals for protein export out of SE (GFP used as reporter) using fluorescence: OD measurements as a ratio of media to cells, normalized by the control (His-GFP-RMR).

FIG. 2E shows a Western blot of an example human protein produced by S. epidermidis, human filaggrin. FIG. 2F-I show two-photon microscopy data characterizing GFP with (H,I) and without (F,G), the RMR cell penetrating polypeptide. 5 μg GFP was added and images were taken at 30 minutes (F,H) or 60 minutes (G,I).

FIG. 3 shows Western blots of mut-IL-4 expressed in E. coli and S. epidermidis.

FIG. 4 is a schematic that shows an overview of Biacore surface plasmon resonance (SPR) for mut-IL-4 activity.

FIG. 5 is a schematic that shows exemplary recombinant constructs and integration in genome.

FIG. 6 is a schematic of an exemplary pUBTR plasmid.

### DETAILED DESCRIPTION OF THE INVENTION

The present disclosure describes, inter alia, one of the first reported examples of commensal skin bacteria that secrete therapeutic proteins to treat skin disease. Additionally, because response to different microbial species and strains will differ between individuals, a modular design to control therapeutic delivery provides considerably improved pharmacokinetics and disease resolution over current approaches that leverage naturally occurring strains.

Before the present invention is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

The articles “a” and “an” are used herein to refer to one or to more than one (i.e. to at least one) of the grammatical object of the article. By way of example, “an element” means one element or more than one element.

The term “including” is used herein to mean, and is used interchangeably with, the phrase “including but not limited to”.

The term “or” is used herein to mean, and is used interchangeably with, the term “and/or,” unless context clearly indicates otherwise.

The term “such as” is used herein to mean, and is used interchangeably, with the phrase “such as but not limited to”.

As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

“Atopic dermatitis” (AD), as used herein, means an inflammatory skin disease characterized by intense pruritus (e.g., severe itch) and by scaly and dry eczematous lesions. The term “atopic dermatitis” includes, but is not limited to, AD caused by or associated with epidermal barrier dysfunction, allergy (e.g., allergy to certain foods, pollen, mold, dust mite, animals, etc.), radiation exposure, and/or asthma. The present disclosure encompasses methods to treat patients with mild, moderate-to-severe or severe AD. As used herein, “moderate-to-severe AD”, is characterized by intensely pruritic, widespread skin lesions that are often complicated by persistent bacterial, viral or fungal infections. Moderate-to-severe AD also includes chronic AD in patients. In many cases, the chronic lesions include thickened plaques of skin, lichenification and fibrous papules. Patients affected by moderate-to-severe AD also, in general, have more than 20% of the body's skin affected, or 10% of skin area in addition to involvement of the eyes, hands and body folds. Moderate-to-severe AD is also considered to be present in patients who require frequent treatment with topical corticosteroids. A patient may also be said to have moderate-to-severe AD when the patient is resistant or refractory to treatment by either a topical corticosteroid or a calcineurin inhibitor or any other commonly used therapeutic agent known in the art. The present disclosure includes methods to treat both the extrinsic and the intrinsic forms of AD. The extrinsic form of AD associated with IgE-mediated sensitization and increased levels of Th2 cytokines involves 70% to 80% of patients with AD. The intrinsic form without IgE-mediated sensitization involves 20% to 30% of patients with AD; these patients have lower levels of IL-4 and IL-13 than extrinsic AD.

As used herein, the terms “carriers”, “carrier system” or “vehicles” refer to compatible substances that are suitable for delivering, containing, or “carrying” a pharmaceutical active ingredient or other materials for administration in a topically applied composition to a patient or subject. Carriers useful herein should be pharmaceutically acceptable. Carriers and vehicles useful herein include any such materials known in the art, which are non-toxic and do not interact with other components of the formulation in which it is contained in a deleterious manner. The term “aqueous” refers to a formulation that contains water or that becomes water-containing following application to the skin or mucosal tissue. Further examples of “carriers” include water, lower alcohols, higher alcohols, polyhydric alcohols, monosaccharides, disaccharides, polysaccharides, hydrocarbon oils, fats and oils, waxes, fatty acids, silicone oils, nonionic surfactants, ionic surfactants, silicone surfactants, and water-based mixtures and emulsion-based mixtures of such carriers.

As used herein, the term “engineered bacterial strain,” or a “recombinant bacterial strain” refers to a strain of bacteria that has been “genetically modified” or “engineered” by the introduction of DNA prepared outside the organism into the bacterial strain. For example, the introduction of a plasmid containing new genes or other nucleic acid sequence(s) into bacteria will allow the bacteria to express those genes or other nucleic acid sequence(s). Alternatively, the plasmid containing new genes or other nucleic acid sequence(s) can be introduced to the bacteria and then integrated into the bacteria's genome, where the bacteria will express those genes or other nucleic acid sequence(s).

As used herein, the term “host cell” is meant to refer to a cell which has been transformed or transfected, or is capable of transformation or transfection by an exogenous polynucleotide sequence.

The term “isolated” for the purposes of the present disclosure designates a biological material (cell, nucleic acid or protein) that has been removed from its original environment (the environment in which it is naturally present). For example, a polynucleotide present in the natural state in a plant or an animal is not isolated, however the same polynucleotide separated from the adjacent nucleic acids in which it is naturally present, is considered “isolated.”

An “isolated nucleic acid molecule” (such as, for example, an isolated promoter) is one which is separated from other nucleic acid molecules which are present in the natural source of the nucleic acid. For example, with regard to genomic DNA, the term “isolated” includes nucleic acid molecules which are separated from the chromosome with which the genomic DNA is naturally associated. Preferably, an “isolated” nucleic acid molecule is free of sequences which naturally flank the nucleic acid molecule in the genomic DNA of the organism from which the nucleic acid molecule is derived.

As used here, the term “genetic element” is meant to refer to a polynucleotide comprising a region that encodes a polypeptide or a polynucleotide region that regulates replication, transcription or translation or other processes important to expression of the polypeptide in a host cell, or a polynucleotide comprising both a region that encodes a polypeptide and a region operably linked thereto that regulates expression. Genetic elements may be comprised within a vector that replicates as an episomal element; that is, as a molecule physically independent of the host cell genome. They may be comprised within plasmids. Genetic elements also may be comprised within a host cell genome; not in their natural state but, rather, following manipulation such as isolation, cloning and introduction into a host cell in the form of purified DNA or in a vector, among others.

As used here, the term “live biotherapeutic product” (or LBP) refers to a product candidate(s) containing bacteria, yeast, and/or other microorganisms.

As used herein, the terms “patient” or “subject”, refers to a human or animal (in the case of an animal, more typically a mammal such as domesticated mammals, or animals such as poultry animals and fish and other seafood or freshwater food creatures), that would be subjected to the treatments and compositions of the present invention.

As used herein, the expression “a subject in need thereof” means a human or non-human animal that exhibits one or more symptoms or indicia of atopic dermatitis, and/or who has been diagnosed with atopic dermatitis. In the context of the present disclosure, “a subject in need thereof” may include, e.g., subjects who, prior to treatment, exhibit (or have exhibited) one or more AD-associated parameters such as, e.g., elevated IGA, BSA, EASI, SCORAD, 5D-Pruritus, and/or NRS score, and/or an elevated level of one or more AD-associated biomarker such as, e.g., IgE and/or TARC (as described elsewhere herein). In certain embodiments, “a subject in need thereof” may include a subset of population which is more susceptible to AD or may show an elevated level of an AD-associated biomarker. In alternate embodiments, the term “subject in need thereof” includes patients with moderate-to-severe AD who have been administered one or more topical corticosteroids (TCS) for more than 6 months, more than 1 year, more than 2 years, more than about 5 years, more than about 7 years, or more than about 10 years. The patients may desire to minimize or avoid the adverse side effects of the TCS.

The term “topical corticosteroids (TCS)”, as used herein includes group I, group II, group III and group IV topical corticosteroids. According to the Anatomical Therapeutic Classification System of World Health Organization, the corticosteroids are classified as weak (group I), moderately potent (Group II) and potent (Group III) and very potent (Group IV), based on their activity as compared to hydrocortisone. Group IV TCS (very potent) are up to 600 times as potent as hydrocortisone and include clobetasol propionate and halcinonide. Group III TCS (potent) are 50 to 100 times as potent as hydrocortisone and include, but are not limited to, betamethasone valerate, betamethasone dipropionate, diflucortolone valerate, hydrocortisone-17-butyrate, mometasone furoate, and methylprednisolone aceponate. Group II TCS (moderately potent) are 2 to 25 times as potent as hydrocortisone and include, but are not limited to, clobetasone butyrate, and triamcinolone acetonide. Group I TCS (mild) includes hydrocortisone.

As used herein, the phrase “pharmaceutically acceptable” refers to those active compounds, materials, engineered bacterial strain or strains, compositions, carriers, and/or dosage forms which are, within the scope of sound medical judgment, suitable for use in contact with the tissues of human beings and animals without excessive toxicity, irritation, allergic response, or other problems or complications, commensurate with a reasonable benefit/risk ratio.

As used herein, the term “polynucleotide(s)” generally refers to any polyribonucleotide or polydeoxyribonucleotide, which may be unmodified RNA or DNA or modified RNA or DNA. Thus, for instance, polynucleotides as used herein refers to, among others, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded, or a mixture of single- and double-stranded regions. In addition, polynucleotide as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term polynucleotide includes DNAs or RNAs as described above that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including simple and complex cells, inter alia. The term polynucleotide also embraces short polynucleotides often referred to as oligonucleotide(s). “Polynucleotide” and “nucleic acid” are often used interchangeably herein.

As used herein, the terms “polypeptide” or “protein” refer to biological molecules, or macromolecules composed of amino-acid residues bonding together in a chain. The definition of polypeptides used herein is intended to encompass proteins (generally higher molecular weight) composed of one or more long chains of amino acid residues and small peptides (generally lower molecular weight) of a few amino acids. In other embodiments, a single amino acid, although not technically a polypeptide, is also considered within the scope of the disclosure.

As used herein, the term “preventing”, refers to completely or almost completely stopping an abnormal skin condition (e.g. AD) from occurring, for example when the patient or subject is predisposed to an abnormal skin condition or at risk of contracting an abnormal skin condition. Preventing can also include inhibiting, i.e. arresting the development, of an abnormal skin condition.

As used herein, a “promoter” is meant to refer to a DNA sequence that directs the transcription of a structural gene. Typically, a promoter is located in the 5′ region of a gene, proximal to the transcriptional start site of a structural gene. If a promoter is an inducible promoter, then the rate of transcription increases in response to an inducing agent. For example, a promoter may be regulated in a tissue-specific manner such that it is only active in transcribing the associated coding region in a specific tissue type(s).

As used herein, the term “reducing the risk of”, refers to lowering the likelihood or probability of an abnormal skin condition (e.g. AD) from occurring, for example when the patient or subject is predisposed to an abnormal skin condition or at risk of contracting an abnormal skin condition.

As used herein, the term “therapeutically effective amount” refers to an amount of a pharmaceutical active compound, or a combination of compounds, or an amount of pharmaceutical active compound delivered by an engineered bacterial strain or strains, for example a skin treatment agent or agents, when administered alone or in combination, to treat, prevent, or reduce the risk of a disease state or condition, for example an abnormal skin condition (e.g. AD). The term also refers to an amount of a pharmaceutical composition containing an active compound or combination of compounds or an engineered bacterial strain or strains that delivers a pharmaceutical active compound. For example, an effective amount refers to an amount of the compound or an amount of the compound delivered by an engineered bacterial strain or strains present in a formulation given to a recipient patient or subject sufficient to elicit biological activity, for example, activity for treating or preventing an abnormal skin condition.

As used herein, the term “treating” refers to providing a therapeutic intervention to cure or ameliorate an abnormal skin condition (e.g. AD).

### Compositions

Described herein are methods of using genetically engineered bacteria to produce therapeutic proteins. Using genetically engineered protein-producing bacteria has several advantages over the prior art method of treating skin disease. Therapeutic proteins are able to treat the underlying cause of defects leading to the skin condition. Further bacteria are able to self-replicate while retaining the inserted gene to continuously produce the therapeutic protein.

The present disclosure provides skin-colonizing bacteria that are genetically altered to express recombinant therapeutic polypeptides for the treatment or prevention of skin disease, and in particular, AD. Using genetically engineered protein-producing bacteria has several advantages over the prior art method of treating skin disease. Therapeutic proteins are able to treat the underlying cause of defects leading to the skin condition. Further, bacteria are able to self-replicate while retaining the inserted gene to continuously produce the therapeutic protein.

The present disclosure provides skin-colonizing bacteria, such as for example, Staphylococcus epidermidis, that are genetically altered to express a mutant IL-4 protein.

### Bacterial Strains

A wide range of bacteria are suitable for use in the present disclosure. Examples include, but are not limited to, non-pathogenic and commensal bacteria. Bacteria suitable for use in the present disclosure include, but are not limited to, Bifidobacterium, Brevibacterium, Cutibacterium (formerly known as Propionibacterium), Lactococcus, Streptococcus, Staphylococcus (e.g., S. epidermidis), Lactobacillus (e.g., L. acidophilus), Pediococcus, Leuconostoc, or Oenococcus.

Certain embodiments involve the use of bacterium Staphylococcus epidermidis. An exemplary strain is S. epidermidis strain ATCC12228. According to some embodiments, S. epidermidis strain NRRL B-4268 is used.

However, other related or similar species found on the skin can also be used.

### Therapeutic Proteins

The present disclosure provides genetically altered microorganisms, e.g., bacteria, capable of expressing recombinant therapeutic proteins. The present disclosure also provides recombinant therapeutic proteins alone (e.g. recombinant therapeutic proteins that are not expressed by a microorganism, e.g., bacteria).

In preferred embodiments of the disclosure, the therapeutic protein comprises a mutant IL-4, or variant thereof. Mutants of IL-4 ([R121D, Y124D]) have been identified that inhibit IL-13/IL-4 responses. R121 and Y124 are both located in the binding pocket of IL-4 to IL-4Ra or IL-13α1. Considerable data have shown that these mutants (1) are antagonists of IL-4R²⁵; (2) inhibit IL-13 dependent or IL-4 dependent CD13 expression in B-cells²⁵ and inhibit B-cell proliferation;²⁶ (3) inhibit IgG4 and IgE synthesis in cultures of PBMN cells;²⁶ and (4) can even prevent antigen-induced allergies in mice.²⁷

The therapeutic protein (and, i.e., the gene from which the protein is expressed) of the disclosure may be from any mammal Non-limiting examples include, but are not limited to, mouse, rat, rabbit, goat, sheep, horse, cow, dog, primate, or human gene sequence. In preferred embodiments of the disclosure, the therapeutic protein comprises a recombinant fusion protein comprising mutant IL-4 operably linked to a cell penetrating protein (CPP). In some embodiments, the CPP is at the C-terminus. In some embodiments, the CPP is at the N-terminus. In some embodiments, a CPP is at both the C- and N-termini. In other embodiments of the disclosure, the therapeutic protein comprises a recombinant fusion protein comprising mutant IL-4 operably linked to an export or secretion signal, which allows the recombinant mutant IL-4 to be exported out of the microorganism (e.g., bacteria). In another embodiment, the therapeutic protein comprises a recombinant fusion protein comprising mutant IL-4 operably linked to a cell penetrating protein (CPP) and to an export or secretion signal.

### Secretion, Export or Signal Peptides

Secretion signals or export signals are peptides on a protein that indicate the protein is destined for the secretory pathway and therefore secreted from the cell. Any secretion signal that facilitates export of a protein out of the S. epidermidis cell and then imported into human keratinocytes is contemplated as a secretion signal. Secretion, export or signal peptides that may be used in the present invention are disclosed in Degering et al. (Applied and Environmental Microbiology, October 2010. P6370-6376), incorporated by reference in its entirety herein.

Non-limiting examples of secretion signals are as follows:

(SEQ ID NO: 1)
MKKLAFAITAASGAAAVLSHHDAEA
(SEQ ID NO: 2)
WLDNRAFSKKFVPVVMATSVALFFLNLAFA
(SEQ ID NO: 3)
MAKKFNYKLPSMVALTLFGTAFTAHQANA
(SEQ ID NO: 4)
MKKRFLSICTMTIAALATTTMVNTSYA
(SEQ ID NO: 5)
NLKKQSKLILIFICIFTFFIMIIQSQFLMG
(SEQ ID NO: 6)
MKIFKLTSLTLAALTLAFPFSHVAQA
(SEQ ID NO: 7)
MKKTVIASTLAVSLGIAGYGLSGHEAHA
(SEQ ID NO: 8)
MKKNKFLVYLLSTALITPTFATQTAFA
(SEQ ID NO: 9)
MKTRQNKYSIRKFSVGASSILIAALLFMGGGSAQA
(SEQ ID NO: 10)
MKNNNETRRFSIRKYTVGVVSIITGITIFVSGQHAQA
(SEQ ID NO: 11)
MKKKLSYMITIMLAFTLSLALGLFFNSAHA

According to some embodiments, the secretion signal protein comprises

(SEQ ID NO: 12)
MKKRFLSICTMTIAALATTTMVNTSYA.

### Cell Penetrating Peptides

A cell penetrating peptide can be used to mediate delivery of cargo in vivo without using any receptors and without causing any significant membrane damage. Cell penetrating peptides that facilitate entry into the skin keratinocytes are contemplated as a cell penetrating peptides of the present disclosure. Non-limiting examples of cell penetrating peptides are as follows:

(SEQ ID NO: 13)
GRKKRRQRRRPPQ;
(SEQ ID NO: 14)
GWTLNSAGYLLGKINLKALAALAKKIL;
(SEQ ID NO: 15)
KLALKLALKALKAALKLA;
(SEQ ID NO: 16)
WEAKLAKALAKALAKHLAKALAKALKACEA;
(SEQ ID NO: 17)
KETWWETWWTEWSQPKKKRKV;
(SEQ ID NO: 18)
RRRRRRRRR;
(SEQ ID NO: 19)
LGTYTQDFNKFHTFPQTAIGVGAP;
(SEQ ID NO: 20)
ACHSALTKHCG;
(SEQ ID NO: 21)
ACKTGSHNQCG;
(SEQ ID NO: 22)
ACTGSTQHQCG;
(SEQ ID NO: 23)
ACTHGQTQSCG;
(SEQ ID NO: 24)
ACSATLQHSCG;
(SEQ ID NO: 25)
ACSLTVNWNCG;
(SEQ ID NO: 26)
ACTSTGRNACG;
(SEQ ID NO: 27)
ACSASTNHNCG;
(SEQ ID NO: 28)
RRWRRWNRFNRRRCR;
(SEQ ID NO: 29)
KLAKLAKKLAKLAKRRWRRWNRFNRRRCR;
(SEQ ID NO: 30)
LVGVFH;
(SEQ ID NO: 31)
DRTTLTN;
(SEQ ID NO: 32)
ACSSSPSKHCG;
(SEQ ID NO: 21)
ACKTGSHNQCG;
(SEQ ID NO: 33)
HIITDPNMAEYL;
(SEQ ID NO: 34)
GPSQPTYPGDDAPVRDLIRFYRDLRRYLNVVTRHRY;
(SEQ ID NO: 35)
WTLNSAGYLLGKINLKALAALAKKIL;
(SEQ ID NO: 36)
YARAAARQARA;
(SEQ ID NO: 37)
KWKLFKKIGAVLKVLTTG;
(SEQ ID NO: 38)
RQIKIWFQNRRMKWKK;
(SEQ ID NO: 39)
YGRKKRRQRRR;
(SEQ ID NO: 40)
ALFLGWLGAAGSTMGAPKKKRKV;
(SEQ ID NO: 41)
TGSTQHQ;
(SEQ ID NO: 42)
ACNAHQARSTCG;
(SEQ ID NO: 43)
WYSCNVCGKAFVLSRHLNRHLRVHRRAT.

### Pro-Peptides

According to some embodiments, the pro-peptide is selected from, but not limited to, the following sequences:

(SEQ ID NO: 44)
LEISSTCDA;
(SEQ ID NO: 45)
LGISSTCNA;
(SEQ ID NO: 46)
LQVDDIPSA;
(SEQ ID NO: 47)
LKISSTCHA;
(SEQ ID NO: 48)
ETANKSNE;
(SEQ ID NO: 49)
ETANESNEGT;
(SEQ ID NO: 50)
ETANASNEGT;
(SEQ ID NO: 51)
ETANKSNAGT;
(SEQ ID NO: 52)
ATANKSNEGT;
(SEQ ID NO: 53)
ETANKSNKGT;
(SEQ ID NO: 54)
KTDTESHNHSSLGTENKNVLD
INSSSHNIKPSQNKSYPS;
(SEQ ID NO: 55)
DTNSDIAKQD;
(SEQ ID NO: 56)
DTTTDIAKQE;
(SEQ ID NO: 57)
DTSADIANQE;
(SEQ ID NO: 58)
GDGEEKGSYAETHGLTADDVESINALNER
ALTLGQPGKPPKELPPSASAPS RAP;
(SEQ ID NO: 59)
ASGGDEEWEG SYAATHGLTA EDVKNINALN
KRALTAGQPG NFPAELPPSA TALFRAPD;
(SEQ ID NO: 60)
ADSGDGAKKG SYAETHGLTA HDVKNINALN
ERAPALGQPG KPPAGAPPFR TPA;
(SEQ ID NO: 61)
ASGDDEEREG SYAETHGLTA EDVKNINALN
KRALTAGQP GNSLTELPPS VSALFRAPD;
(SEQ ID NO: 62)
DNGAGEETKS YAETYRLTAD DVANINALNE
SAPAASSAGP SFRAP;
(SEQ ID NO: 63)
ADAGDGAKEGSYAETHGLTAHDVKNINAL
NERALAVGQPGKPPAGAPPFR TPA
(SEQ ID NO: 64)
ASRGDGEWEGSYAETHGLTAEDVKNI
NALNKRALTAGQPGNSAAELPPSA
SALFRAPD.

According to some embodiments, the pro-peptide comprises

(SEQ ID NO: 54)
KTDTESHNHSSLGTENKNVLDINSSSHNIKPSQNKSYPS

### Genetic Construct

The disclosure utilizes standard molecular biology techniques, e.g., those described in (Sambrook et al. 2001).

Functional genetic analyses of common skin colonizers S. aureus and S. epidermidis have previously been limited due to the presence of Type I and IV restriction systems in virtually all strains of these bacteria. These restriction systems recognize methylated cytosine bases in DNA from standard clone expansion systems such as DH10B E. coli. Using a methylation deficient E. coli strain, NEB dam⁻/dcm⁻ as an intermediated host, similar to reported DC10B,⁴⁵ several constructs have been created and transformed in S. epidermidis strain NRRL B-4268, which has the same origination as ATCC 12228,⁴⁶ a commensal, non-pathogenic isolate lacking ica operons implicated in S. epidermidis-associated catheter bloodstream infections.

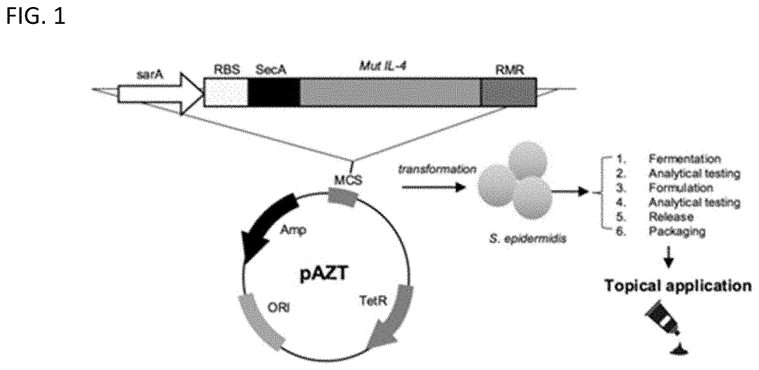

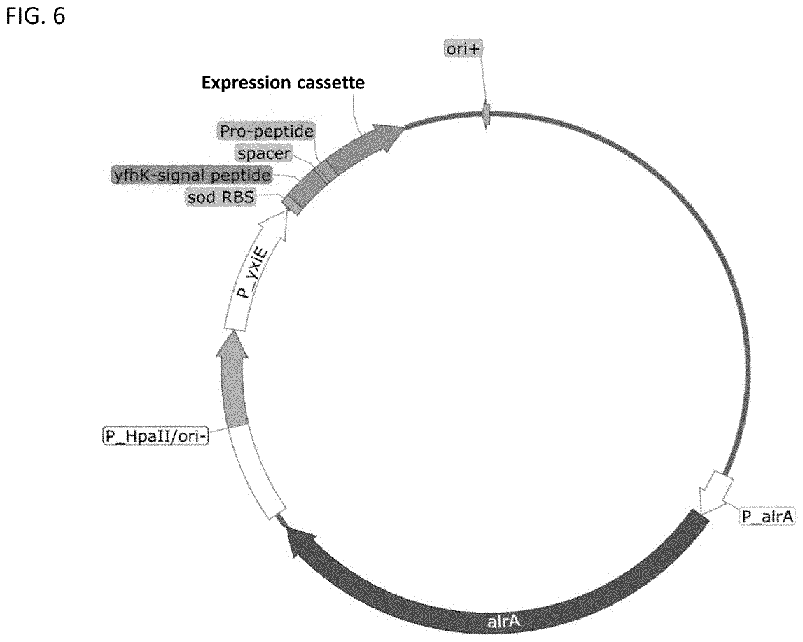

A general plasmid (FIG. 1) has been developed that can encode a protein that is exported out of the S. epidermidis cell and then imported into human keratinocytes. This plasmid, pAZT, is based on pJB38,⁴⁷ an allelic exchange E. coli-staphylococcal shuttle vector, and we have improved on its functionality. According to some embodiments, the plasmid is a pUBTR plasmid. A schematic of an exemplary pUBTR plasmid is shown in FIG. 6. The engineered S. epidermidis effectively colonizes reconstituted epidermis and is capable of producing up to 30-44 μg of protein per ˜10⁸ CFU/mL and secreted protein in the media is approximately 1-2 μg/mL. Additional features include colonization on mouse skin, optimization of promoter selection, evidence of a functional secretion signal, ability to produce human proteins, and ability to deliver protein through the stratum cornuem.

According to some embodiments, the promoter is selected from, but not limited to, the following promoters: sarAP1 (S. aureus; see, e.g., Malone, C. L. et al. Fluorescent reporters for Staphylococcus aureus. Journal of Microbiological Methods 77, 251-260), pPS4 (S. epidermidis; see, e.g., Manna, A. C. et al. Journal of Bacteriology 180, 3828-3836 (1998)), δ-hemolysin (S. epidermidis; see, e.g., Franke, G. C. et al. Journal of Microbiological Methods 71, 123-132), XylR (S. epidermidis; see, e.g., Bhaysar, A. P., Zhao, X. & Brown, E. D. Applied and environmental microbiology 67, 403-410), PyxiE (B. subtilus) and PespA (S. epidermidis; GenBank: AE015929.1 for ATCC12228).

According to some embodiments, the promoter in an ESP promoter comprising the following sequence. The underlined portion shows the RBS.

(SEQ ID NO: 65)
ATGTTTTCACTCTTTTTTTATTTATTTGAGCGC
TAATTGTTATTTATCATTTGATAGTCTCAAAAA
TTTTATTAATGAGTAAGTTACTAATAATTGTGT
CCAAATTCTTATAAAAAATATTTTAAAAAATAA
ATATAATATTTAATATAATGTAAACAGATAGGG
TTTTTATTTTAACATTTAGGCATTTAATCACAT
ATTTTATCAGTTTATATATATAATTTAATTGTA
ACAGCTGTTATTAAAAAAATAAATTTTATAATT
TTTGGAGGTTATCAT

As described herein, the bacteria species selected for the composition is transformed using known recombinant techniques to express a protein of interest.

### Formulations

A formulation for use according to the present disclosure may comprise any pharmaceutically effective amount of a genetically engineered microorganism, e.g., bacteria, to produce a therapeutically effective amount of a desired polypeptide, for example, at least about 0.01%, about 0.05%, about 0.1%, about 0.2%, about 0.3%, about 0.4%, about 0.5%, about 0.6%, about 0.7%, about 0.8%, about 0.9%, about 1.0%, about. 1.5%, about 2.0%, about 3.0%, about 4.0%, about 5.0%, about 6.0%, about 7.0%, about 8.0%, about 9.0%, about 10.0%, about 11.0%, about 12.0%, about 13.0%, about 14.0%, about 15.0%, about 16.0%, about 17.0%, about 18.0%, about 19.0%, about 20.0%, about 25.0%, about 30.0%, about 35.0%, about 40.0%, about 45.0%, about 50.0% or more by weight of the genetically engineered microorganism, e.g., bacteria, the upper limit of which is about 90.0% by weight of the genetically engineered microorganism, e.g., bacteria.

In an alternative embodiment the formulations for use according to the present disclosure can comprise, for example, at least about 0.01% to about 30%, about 0.01% to about 20%, about 0.01% to about 5%, about 0.1% to about 30%, about 0.1% to about 20%, about 0.1% to about 15%, about 0.1% to about 10%, about 0.1% to about 5%, about 0.2% to about 5%, about 0.3% to about 5%, about 0.4% to about 5%, about 0.5% to about 5%, about 1% to about 5%, or more by weight of recombinant bacteria.

The topical formulation for use in the present disclosure can be in any form suitable for application to the body surface, such as a cream, lotion, sprays, solution, gel, ointment, paste, plaster, paint, bioadhesive, suspensions, emulsions, or the like, and/or can be prepared so as to contain liposomes, micelles, and/or microspheres. Such a formulation can be used in combination with an occlusive overlayer so that moisture evaporating from the body surface is maintained within the formulation upon application to the body surface and thereafter. The formulation can include a living cell culture composition and can comprise at least one engineered bacterial strain that produces a recombinant polypeptide. This engineered living cell culture composition can deliver the polypeptide directly to the skin for treating or preventing abnormal skin conditions.

Topical formulations include those in which any other active ingredient(s) is (are) dissolved or dispersed in a dermatological vehicle known in the art (e.g. aqueous or non-aqueous gels, ointments, water-in-oil or oil-in-water emulsions). Constituents of such vehicles can comprise water, aqueous buffer solutions, non-aqueous solvents (such as ethanol, isopropanol, benzyl alcohol, 2-(2-ethoxyethoxy)ethanol, propylene glycol, propylene glycol monolaurate, glycofurol or glycerol), oils (e.g. a mineral oil such as a liquid paraffin or petrolatum, natural or synthetic triglycerides such as Miglyol™ or Neobee®, fully saturated, partially saturated or unsaturated long chain fatty acids or fatty alcohols, or silicone oils such as dimethicone), waxes (very long chain fatty acids and alcohols) and emulsifying agents (polysorbates). Depending, inter alia, upon the nature of the formulation as well as its intended use and site of application, the dermatological vehicle employed can contain one or more components (for example, when the formulation is an aqueous gel, components in addition to water) selected from the following list: a solubilizing agent or solvent (e.g. a β-cyclodextrin, such as hydroxypropyl β-cyclodextrin, or an alcohol or polyol such as ethanol, propylene glycol or glycerol); a thickening agent (e.g. hydroxyethylcellulose, hydroxypropylcellulose, carboxymethylcellulose or carbomer); a gelling agent (e.g. a polyoxyethylene-polyoxypropylene copolymer); a preservative (e.g. benzyl alcohol, benzalkonium chloride, chlorhexidine, chlorbutol, a benzoate, potassium sorbate or EDTA or salt thereof); and pH buffering agent(s) (such as a mixture of dihydrogen phosphate and hydrogen phosphate salts, or a mixture of citric acid and a hydrogen phosphate salt).

A pharmaceutically acceptable carrier can also be incorporated in the formulation of the present disclosure and can be any carrier conventionally used in the art. Examples thereof include water, lower alcohols, higher alcohols, polyhydric alcohols, monosaccharides, disaccharides, polysaccharides, hydrocarbon oils, fats and oils, waxes, fatty acids, silicone oils, nonionic surfactants, ionic surfactants, silicone surfactants, and water-based mixtures and emulsion-based mixtures of such carriers.

According to some embodiments, a pharmaceutically acceptable antioxidant can be incorporated to preserve the bacteria from deterioration. These can include, but are not limited to, ascorbic acid (vitamin C), α-tocopherol (Vitamin E), β-tocopherol, γ-tocopherol, δ-tocopherol, ε-tocopherol, ζι-tocopherol, ζ2-tocopherol, η-tocopherol, and retinol (Vitamin A) as well as derivatives thereof, e.g., esters.

The term “pharmaceutically acceptable” or “pharmaceutically acceptable carrier” is used herein to refer to a compound or composition that can be incorporated into a pharmaceutical formulation without causing undesirable biological effects or unwanted interaction with other components of the formulation. “Carriers” or “vehicles” as used herein refer to carrier materials suitable for incorporation in a topically applied composition. Carriers and vehicles useful herein include any such materials known in the art, which are non-toxic and do not interact with other components of the formulation in which it is contained in a deleterious manner. The term “aqueous” refers to a formulation that contains water or that becomes water-containing following application to the skin or mucosal tissue.

A film former, when it dries, forms a protective film over the site of application. The film inhibits removal of the active ingredient and keeps it in contact with the site being treated.

An example of a film former that is suitable for use in this disclosure is Flexible Collodion, USP. As described in Remington: The Science and Practice of Pharmacy, 19th Ed. (Easton, Pa.: Mack Publishing Co., 1995), at page 1530, collodions are ethyl ether/ethanol solutions containing pyroxylin (a nitrocellulose) that evaporate to leave a film of pyroxylin. A film former can act additionally as a carrier. Solutions that dry to form a film are sometimes referred to as paints. Creams, as is well known in the arts of pharmaceutical formulation, are viscous liquids or semisolid emulsions, either oil-in-water or water-in-oil.

Cream bases are water-washable, and contain an oil phase, an emulsifier, and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic or amphoteric surfactant.

Lotions are preparations to be applied to the skin surface without friction and are typically liquid or semiliquid preparations in which particles, including the active agent, are present in a water or alcohol base. Lotions are usually suspensions of solids, and preferably, comprise a liquid oily emulsion of the oil-in-water type. Lotions are preferred formulations herein for treating large body areas, because of the ease of applying a more fluid composition. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethyl-cellulose, or the like.

Solutions are homogeneous mixtures prepared by dissolving one or more chemical substances (solutes) in a liquid such that the molecules of the dissolved substance are dispersed among those of the solvent. The solution can contain other pharmaceutically or cosmetically acceptable chemicals to buffer, stabilize or preserve the solute. Common examples of solvents used in preparing solutions are ethanol, water, propylene glycol or any other acceptable vehicles. As is of course well known, gels are semisolid, suspension-type systems. Single-phase gels contain organic macromolecules distributed substantially uniformly throughout the carrier liquid, which is typically aqueous, but also, preferably, contain an alcohol, and, optionally, an oil. Preferred “organic macromolecules,” i.e., gelling agents, are cross-linked acrylic acid polymers such as the “carbomer” family of polymers, e.g., carboxypolyalkylenes that can be obtained commercially under the Carbopol trademark. Also preferred are hydrophilic polymers such as polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, and methylcellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof. Ointments, as also well known in the art, are semisolid preparations that are typically based on petrolatum or other petroleum derivatives. The specific ointment base to be used, as will be appreciated by those skilled in the art, is one that will provide for a number of desirable characteristics, e.g., emolliency or the like. As with other carriers or vehicles, an ointment base should be inert, stable, nonirritating, and nonsensitizing. As explained in Remington: The Science and Practice of Pharmacy, 19^{th} Ed. (Easton, Pa.: Mack Publishing Co., 1995), at pages 1399-1404, ointment bases can be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin, and hydrophilic petrolatum.

Emulsion ointment bases are either water-in-oil (W/O) emulsions or oil-in-water (O/W) emulsions, and include, for example, acetyl alcohol, glyceryl monostearate, lanolin, and stearic acid. Preferred water-soluble ointment bases are prepared from polyethylene glycols of varying molecular weight; see Remington: The Science and Practice of Pharmacy for further information.

Pastes are semisolid dosage forms in which the active agent is suspended in a suitable base. Depending on the nature of the base, pastes are divided between fatty pastes or those made from single-phase aqueous gels. The base in a fatty paste is generally petrolatum or hydrophilic petrolatum or the like. The pastes made from single-phase aqueous gels generally incorporate carboxymethylcellulose or the like as a base.

Enhancers are those lipophilic co-enhancers typically referred to as “plasticizing” enhancers, i.e., enhancers that have a molecular weight in the range of about 150 to 1000, an aqueous solubility of less than about 1 wt. %, preferably less than about 0.5 wt. %, and most preferably less than about 0.2 wt. %. The Hildebrand solubility parameter δ of plasticizing enhancers is in the range of about 2.5 to about 10, preferably in the range of about 5 to about 10. Preferred lipophilic enhancers are fatty esters, fatty alcohols, and fatty ethers. Examples of specific and most preferred fatty acid esters include methyl laurate, ethyl oleate, propylene glycol monolaurate, propylene glycerol dilaurate, glycerol monolaurate, glycerol monooleate, isopropyl n-decanoate, and octyldodecyl myristate. Fatty alcohols include, for example, stearyl alcohol and oleyl alcohol, while fatty ethers include compounds wherein a diol or triol, preferably a C2-C4 alkane diol or triol, are substituted with one or two fatty ether substituents.

Additional permeation enhancers will be known to those of ordinary skill in the art of topical drug delivery, and/or are described in the pertinent texts and literature. See, e.g., Percutaneous Penetration Enhancers, eds. Smith et al. (CRC Press, 1995)(incorporated herein by reference).

Various other additives can be included in the compositions of the present disclosure in addition to those identified above. These include, but are not limited to, antioxidants, astringents, perfumes, preservatives, emollients, pigments, dyes, humectants, propellants, and sunscreen agents, as well as other classes of materials whose presence can be pharmaceutically or otherwise desirable. Typical examples of optional additives for inclusion in the formulations of the disclosure are as follows: preservatives such as sorbate; solvents such as isopropanol and propylene glycol; astringents such as menthol and ethanol; emollients such as polyalkylene methyl glucosides; humectants such as glycerine; emulsifiers such as glycerol stearate, PEG-100 stearate, polyglyceryl-3 hydroxylauryl ether, and polysorbate 60; sorbitol and other polyhydroxyalcohols such as polyethylene glycol; sunscreen agents such as octyl methoxyl cinnamate (available commercially as Parsol MCX) and butyl methoxy benzoylmethane (available under the tradename Parsol 1789); antioxidants such as ascorbic acid (Vitamin C), α-tocopherol (Vitamin E), β-tocopherol, γ-tocopherol, δ-tocopherol, ε-tocopherol, ζι-tocopherol, ζ2-tocopherol, η-tocopherol, and retinol (Vitamin A); essential oils, ceramides, essential fatty acids, mineral oils, vegetable oils (e.g., soya bean oil, palm oil, liquid fraction of shea butter, sunflower oil), animal oils (e.g., perhydrosqualene), synthetic or semi-synthetic oils (e.g., squalene), silicone oils or waxes (e.g., cyclomethicone and dimethicone), fluorinated oils (generally perfluoropolyethers), fatty alcohols (e.g., cetyl alcohol), and waxes (e.g., beeswax, carnauba wax, and paraffin wax); skin-feel modifiers; and thickeners and structurants such as swelling clays and cross-linked carboxypolyalkylenes that can be obtained commercially under the Carbopol trademark. Other additives include beneficial agents such as those materials that condition the skin (particularly, the upper layers of the skin in the stratum corneum) and keep it soft by retarding the decrease of its water content and/or protect the skin. Such conditioners and moisturizing agents include, by way of example, pyrrolidine carboxylic acid and amino acids; organic antimicrobial agents such as 2,4,4′-10 trichloro-2-hydroxy diphenyl ether (triclosan) and benzoic acid; anti-inflammatory agents such as acetylsalicylic acid and glycyrrhetinic acid; anti-seborrheic agents such as retinoic acid; vasodilators such as nicotinic acid; inhibitors of melanogenesis such as kojic acid; and mixtures thereof. Further additional active agents including, for example, alpha hydroxyacids, alpha ketoacids, polymeric hydroxyacids, moisturizers, collagen, marine extract, and antioxidants such as ascorbic acid (Vitamin C), α-tocopherol (Vitamin E), β-tocopherol, γ-tocopherol, δ-tocopherol, ε-tocopherol, ζι-tocopherol, ζ2-tocopherol, η-tocopherol, and retinol (Vitamin A), and/or pharmaceutically acceptable salts, esters, amides, or other derivatives thereof. A preferred tocopherol compound is a-tocopherol. Additional agents include those that are capable of improving oxygen supply in skin tissue, as described, for example, in Gross, et al, WO 94/00098 and Gross, et al, WO 94/00109, both assigned to Lancaster Group AG (incorporated herein by reference). Sunscreens and UV absorbing compounds can also be included. Non-limiting examples of such sunscreens and UV absorbing compounds include aminobenzoic acid (PABA), avobenzone, cinoxate, dioxybenzone, homosalate, menthyl anthranilate, oxtocrylene, octyl methoxycinnamate, octyl salicylate, oxybenzone, padimate O, phenylbenzimidazole sulfonic acid, sulisobenzone, titanium dioxide, trolamine salicylate, zinc oxide, ensulizole, meradimate, octinoxate, octisalate, and octocrylene. See Title 21. Chapter 1. Subchapter D. Part 352. “Sunscreen drug products for over-the-counter human use” incorporated herein in its entirety.

Other embodiments can include a variety of non-carcinogenic, non-irritating healing materials that facilitate treatment with the formulations of the disclosure. Such healing materials can include nutrients, minerals, vitamins, electrolytes, enzymes, herbs, plant extracts, glandular or animal extracts, or safe therapeutic agents that can be added to the formulation to facilitate the healing of dermal disorders.

The amounts of these various additives are those conventionally used in the cosmetics field, and range, for example, from about 0.01% to about 20% of the total weight of the topical formulation.

The formulations of the disclosure can also include conventional additives such as opacifiers, fragrance, colorant, stabilizers, surfactants, and the like. In certain embodiments, other agents can also be added, such as antimicrobial agents, to prevent spoilage upon storage, i.e., to inhibit growth of microbes such as yeasts and molds. Suitable antimicrobial agents are typically selected from the group consisting of the methyl and propyl esters of p-hydroxybenzoic acid (i.e., methyl and propyl paraben), sodium benzoate, sorbic acid, imidurea, and combinations thereof. In other embodiments, other agents can also be added, such as repressors and inducers, i.e., to inhibit (i.e. glycose) or induce (i.e. xylose) the production of the polypeptide of interest. Such additives can be employed provided they are compatible with and do not interfere with the function of the formulations.

The formulations can also contain irritation-mitigating additives to minimize or eliminate the possibility of skin irritation or skin damage resulting from the chemical entity to be administered, or other components of the composition.

Suitable irritation-mitigating additives include, for example: a-tocopherol; monoamine oxidase inhibitors, particularly phenyl alcohols such as 2-phenyl-1-ethanol; glycerin; salicylates; ascorbates; ionophores such as monensin; amphiphilic amines; ammonium chloride; N-acetylcysteine; and/or capsaicin. The irritation-mitigating additive, if present, can be incorporated into the compositions at a concentration effective to mitigate irritation or skin damage, typically representing not more than about 20 wt. %, more typically not more than about 5 wt. %, of the formulation.

Further suitable pharmacologically active agents that can be incorporated into the present formulations in certain embodiments and thus topically applied along with the active agent include, but are not limited to, the following: agents that improve or eradicate pigmented or non-pigmented age spots, keratoses, and wrinkles; antimicrobial agents; antibacterial agents; antipruritic and antixerotic agents; anti-inflammatory agents; local anesthetics and analgesics; corticosteroids; retinoids; vitamins; hormones; and antimetabolites.

Some examples of topical pharmacologically active agents include acyclovir, amphotericins, chlorhexidine, clotrimazole, ketoconazole, econazole, miconazole, metronidazole, minocycline, nystatin, neomycin, kanamycin, phenytoin, para-amino benzoic acid esters, octyl methoxycinnamate, octyl salicylate, oxybenzone, dioxybenzone, tocopherol, tocopheryl acetate, selenium sulfide, zinc pyrithione, diphenhydramine, pramoxine, lidocaine, procaine, erythromycin, tetracycline, clindamycin, crotamiton, hydroquinone and its monomethyl and benzyl ethers, naproxen, ibuprofen, cromolyn, retinol, retinyl palmitate, retinyl acetate, coal tar, griseofulvin, estradiol, hydrocortisone, hydrocortisone 21-acetate, hydrocortisone 17-valerate, hydrocortisone 17-butyrate, progesterone, betamethasone valerate, betamethasone dipropionate, triamcinolone acetonide, fluocinonide, clobetasol propionate, minoxidil, dipyridamole, diphenylhydantoin, benzoyl peroxide, and 5-fluorouracil.

A cream, lotion, gel, ointment, paste or the like can be spread on the affected surface and gently rubbed in. A solution can be applied in the same way, but more typically will be applied with a dropper, swab, or the like, and carefully applied to the affected areas.

The application regimen will depend on a number of factors that can readily be determined, such as the severity of the condition and its responsiveness to initial treatment, but will normally involve one or more applications per day on an ongoing basis. One of ordinary skill can readily determine the optimum amount of the formulation to be administered, administration methodologies and repetition rates. In general, it is contemplated that the formulations of the disclosure will be applied in the range of once or twice weekly up to once or twice daily. In certain embodiments, the formulations of the disclosure are applied no more than once daily.

### Methods

The disclosure provides methods for treating a skin disease, wherein the methods comprise administering to a subject in need of such treatment a genetically engineered microorganism, e.g., genetically engineered bacteria, capable of expressing a recombinant therapeutic fusion protein of the disclosure, thereby treating the subject. In a preferred embodiment, the disease is atopic dermatitis. In some embodiments, the skin condition is atopic dermatitis (AD). In a further embodiment, the AD is moderate-to-severe AD.

In yet another preferred embodiment, the recombinant therapeutic fusion protein comprises a mutant IL-4 as described herein. In other embodiments, the recombinant therapeutic fusion protein comprises a mutant IL-4 as described herein operably linked to a cell penetrating peptide. In further embodiments, the recombinant therapeutic fusion protein is operably linked to an export signal.

In some embodiments, the method in an improvement in an AD-associated parameter, wherein the improvement in the AD-associated parameter is selected from the group consisting of (a) a decrease from baseline in Investigator's Global Assessment (IGA) score; (b) a decrease from baseline in Body Surface Area Involvement of Atopic Dermatitis (BSA) score; (c) a decrease from baseline in Eczema Area and Severity Index (EASI) score; (d) a decrease from baseline in SCORAD score; (e) a decrease from baseline in 5-D Pruritus Scale; and (f) a decrease from baseline in Pruritus Numeric Rating Scale (NRS) score.

Examples of “AD-associated parameters” include: (a) Investigators Global Assessment (IGA); (b) Body Surface Area Involvement of Atopic Dermatitis (BSA); (c) Eczema Area and Severity Index (EASI); (d) SCORAD; (e) 5-D Pruritus Scale; and (f) Pruritus Numeric Rating Scale (NRS). An “improvement in an AD-associated parameter” means a decrease from baseline of one or more of IGA, BSA, EASI, SCORAD, 5-D Pruritus Scale, or NRS. As used herein, the term “baseline,” with regard to an AD-associated parameter, means the numerical value of the AD-associated parameter for a subject prior to or at the time of administration of a pharmaceutical composition of the present disclosure.

To determine whether an AD-associated parameter has “improved,” the parameter is quantified at baseline and at one or more time points after administration of the pharmaceutical composition of the present disclosure. For example, an AD-associated parameter may be measured at day 1, day 2, day 3, day 4, day 5, day 6, day 7, day 8, day 9, day 10, day 11, day 12, day 14, day 15, day 22, day 25, day 29, day 36, day 43, day 50, day 57, day 64, day 71, day 85; or at the end of week 1, week 2, week 3, week 4, week 5, week 6, week 7, week 8, week 9, week 10, week 11, week 12, week 13, week 14, week 15, week 16, week 17, week 18, week 19, week 20, week 21, week 22, week 23, week 24, or longer, after the initial treatment with a pharmaceutical composition of the present disclosure. The difference between the value of the parameter at a particular time point following initiation of treatment and the value of the parameter at baseline is used to establish whether there has been an “improvement” (e.g., a decrease) in the AD associated parameter.

Investigator's Global Assessment (IGA). The IGA is an assessment scale used in clinical settings to determine the severity of AD and clinical response to treatment based on a 6-point scale ranging from 0 (clear) to 5 (very severe). According to certain embodiments of the present disclosure, administration of the cell culture composition to a patient results in a decrease in IGA score. For example, the present disclosure includes therapeutic methods which result in a decrease from baseline in IGA score of at least about 10%, 15%, 20%, 25%, 30%, 35%, 40%, 45%, 50%, 55%, 60%, 65%, 70%, 75%, or more at day 4, 8, 15, 22, 25, 29, 36, 43, 50, 57, 64, 71, 85 or later following administration of the cell culture composition.

Body Surface Area Involvement of Atopic Dermatitis (BSA). BSA is assessed for each major section of the body (head, trunk, arms and legs) and is reported as a percentage of all major body sections combined. According to certain embodiments of the present disclosure, administration of the cell culture composition to a patient results in a decrease in BSA score. For example, the present disclosure includes therapeutic methods which result in a decrease from baseline in BSA score of at least about 10%, 15%, 20%, 25%, 30%, 35%, 40%, 45%, 50%, 55%, 60%, 65%, 70%, 75% or more at day 4, 8, 15, 22, 25, 29, 36, 43, 50, 57, 64, 71, 85 or later following administration of the cell culture composition.

Eczema Area and Severity Index (EASI). The EASI is a validated measure used in clinical settings to assess the severity and extent of AD. (Hanifin et al. 2001, Exp. Dermatol. 70:1 1-18). Four AD disease characteristics are assessed for severity by a physician or other qualified medical professional on a scale of 0 (absent) through 3 (severe). In addition, the area of AD involvement is assessed as a percentage by body area of head, trunk, arms and legs and converted to a score of 0 to 6. According to certain embodiments of the present disclosure, administration of the cell culture composition to a patient results in a decrease in EASI score. For example, the present disclosure includes therapeutic methods which result in a decrease from baseline in EASI score of at least about 10%, 15%, 20%, 25%, 30%, 35%, 40%, 45%, 50%, 55%, 60%, 65%, 70%, 75% or more at day 4, 8, 15, 22, 25, 29, 36, 43, 50, 57, 64, 71, 85 or later following administration of the cell culture composition.

SCORAD. SCORing Atopic Dermatitis (SCORAD) is a clinical assessment of the severity (e.g., extent or intensity) of atopic dermatitis developed by the European Task Force on Atopic Dermatitis (Consensus Report of the European Task Force on Atopic Dermatitis, 1993, Dermatology (Basel) 786(1):23-31). The extent of AD is assessed as a percentage of each defined body area and reported as the sum of all areas, with a maximum score of 100% (assigned as “A” in the overall SCORAD calculation). The severity of 6 specific symptoms of AD is assessed using the following scale: none (0), mild (1), moderate (2), or severe (3) (for a maximum of 18 total points, assigned as “B” in the overall SCORAD calculation). Subjective assessment of itch and sleeplessness is recorded for each symptom by the patient or relative on a visual analogue scale (VAS), where 0 is no itch (or sleeplessness) and 10 is the worst imaginable itch (or sleeplessness), with a maximum possible score of 20. This parameter is assigned as “C” in the overall SCORAD calculation. The SCORAD is calculated as: A 5+7B/2+C. According to certain embodiments of the present disclosure, administration of an IL-4R antagonist to a patient results in a decrease in SCORAD score. For example, the present disclosure includes therapeutic methods which result in a decrease from baseline in SCORAD of at least about 10%, 15%, 20%, 25%, 30%, 35%, 40%, 45%, 50%, 55%, 60%, 65%, 70%, 75% or more at day 4, 8, 15, 22, 25, 29, 36, 43, 50, 57, 64, 71, 85 or later following administration of the cell culture composition.

5-D Pruritus Scale. The 5-D Pruritus Scale is a 1-page, 5-question tool used in clinical settings to assess 5 dimensions of background itch: degree, duration, direction, disability, and distribution. (Elman and Hynan, 2010, Brit. J. Dermatol. 162:587-593). Each question corresponds to 1 of the 5 dimensions of itch; patients rate their symptoms as “present” or on a 1 to 5 scale, with 5 being the most affected. According to certain embodiments of the present disclosure, administration of an IL-4R antagonist to a patient results in a decrease in 5-D Pruritus Scale. For example, the present disclosure includes therapeutic methods which result in a decrease from baseline in 5-D Pruritus Scale of at least about 10%, 15%, 20%, 25%, 30%, 35%, 40%, 45%, 50%, 55%, 60%, 65%, 70%, 75% or more at day 4, 8, 15, 22, 25, 29, 36, 43, 50, 57, 64, 71, 85 or later following administration of the cell culture composition.

Pruritus Numeric Rating Scale (NRS). The Pruritus NRS is a single-question assessment tool that is used to assess a subject's worst itch, on a scale of 1 to 10, as a result of AD in the previous 12 hours. According to certain embodiments of the present disclosure, administration of an IL-4R antagonist to a patient results in a decrease in NRS score. For example, the present disclosure includes therapeutic methods which result in a decrease from baseline in NRS score of at least about 10%, 15%, 20%, 25%, 30%, 35%, 40%, 45%, 50%, 55%, 60%, 65%, 70%, 75% or more at the end of week 1, 2, 3, 4, 5, 6, 7, 8, 9, 10, 11, 12, or later following administration of the cell culture composition.

Global Individual Signs Score (GISS). Individual components of the AD lesions (erythema, infiltration/population, excoriations, and lichenification) is rate globally (i.e., each assessed for the whole body, not by anatomical region) on a 4-point scale (from 0=none to 3=severe) using the EASI severity grading criteria.

Pruritus Categorical Scale. The Pruritus categorical scale is a 4-point scale used to assess symptoms that has been used in clinical studies of AD and has less of a “middling” effect (Kaufmann 2006). The scale is rated as follows: 0: absence of pruritus; 1: mild, pruritus (occasional slight itching/scratching); 2: moderate pruritus (constant or intermittent itching/scratching that does not disturb sleep) and 3: severe pruritus (bothersome itching/scratching that disturbs sleep).

Patient Oriented Eczema Measure (POEM). The POEM is a 7-item, validated questionnaire used in clinical practice and clinical trials to assess disease symptoms in children and adults (Charman 2004). The format is a response to 7 items (dryness, itching, flaking, cracking, sleep loss, bleeding, and weeping) with a scoring system of 0 to 28; a high score is indicative of a poor QOL.

Dermatology Life Quality Index (DLQI). The DLQI is a 10-item, validated questionnaire used in clinical practice and clinical trials to assess the impact of AD disease symptoms and treatment on QOL (Badia 1999). The format is a simple response to 10 items, which assess QOL over the past week, with an overall scoring system of 0 to 30; a high score is indicative of a poor QOL.

Itchy QOL. Itchy QOL is a validated pruritus-specific instrument that addresses the symptom, emotional, and functional impact of pruritus. There is an overall score as well as subscale scores to address the 3 types of impact. This is a reliable, valid, and responsive questionnaire (Desai 2008). [0126] EQ-5D. The EQ-5D is a standardized measure of health status developed by the EuroQOI Group in order to provide a simple, generic measure of health for clinical and economic appraisal. The EQ-5D as a measure of health related QOL, defines health in terms of 5 dimensions: mobility, self-care, usual activities, pain/discomfort, and anxiety/depression. Each dimension has 3 ordinal levels of severity: “no problem” (1), “some problems” (2), “severe problems” (3). Overall health state is defined as a 5-digit number. Health states defined by the 5-dimensional classification can be converted into corresponding index scores that quantify health status, where 0 represents “death” and 1 represents “perfect health.”

Patient Global Assessment of Disease Status and Treatment Effect. Patients rate their overall wellbeing based on a 5-point Likert scale from poor to excellent. Patients are asked: “Considering all the ways in which your eczema affects you, indicate how well you are doing”. Response choices are: “Poor”; “Fair”; “Good”; “Very Good”; “Excellent”. For treatment effect, patients rate their satisfaction with the study treatment based on a 5-point Likert scale from poor to excellent. Patients are asked: “How would you rate the way your eczema responded to the study medication?” Response choices are: “Poor”; “Fair”; “Good”; “Very Good”; “Excellent”.

In some embodiments, the subject exhibits an elevated level of at least one AD-associated biomarker prior to, or at the time of treatment. An “AD-associated biomarker” includes a biomarker associated with Type 2 helper T-cell (Th2)-driven inflammation. Exemplary AD-associated biomarkers include, but are not limited to, e.g., thymus and activation-regulated chemokine (TARC; also known as CCL17), immunoglobulin E (IgE), eotaxin-3 (also known as CCL26), lactate dehydrogenase (LDH), eosinophils, antigen-specific IgE (e.g., Phadiatop™ test), and periostin. The term “AD-associated biomarker” also includes a gene or gene probe known in the art which is differentially expressed in a subject with AD as compared to a subject without AD. For example, genes which are significantly up-regulated in a subject with AD include, but are not limited to, T-helper 2 (Th2)-associated chemokines such as CCL13, CCL17, CCL18 and CCL26, markers of epidermal proliferation such as K16, Ki67, and T-cell and dendritic cell antigens CD2, CD1 b, and CD1 c (Tintle et al. 201 1; J. Allergy Clin. Immunol. 128: 583-593). Alternatively, “AD-associated biomarker” also includes genes which are down regulated due to AD such as terminal differentiation proteins (e.g., loricrin, filaggrin and involucrin) (Tintle et al. 201 1; J.Allergy Clin. Immunol. 128: 583-593). Certain embodiments of the disclosure pertain to use of these biomarkers for monitoring disease reversal with the administration of the IL-4R antagonist. Methods for detecting and/or quantifying such AD-associated biomarkers are known in the art; kits for measuring such AD-associated biomarkers are available from various commercial sources; and various commercial diagnostic laboratories offer services which provide measurements of such biomarkers as well.

According to certain embodiments, methods for treating AD are provided which comprise: selecting a subject who exhibits a level of at least one AD-associated biomarker prior to or at the time of treatment which signifies the disease state; and administering to the subject a pharmaceutical composition comprising a cell culture composition as described herein. In certain embodiments, the patient is selected by determining if the level of an AD-associated biomarker is elevated. The level of an AD-associated biomarker is determined or quantified by acquiring a sample from the patient for a biomarker assay known in the art. In certain other embodiments, a patient is selected by acquiring information relating to an elevated level of an AD-associated biomarker from the patient.

In certain embodiments, the present disclosure includes methods to treat patients who have AD for more than 1 year, more than about 5 years, more than about 10 years, or more than about 15 years.

In other embodiments, the subject is resistant, non-responsive or inadequately responsive to treatment by either a topical corticosteroid (TCS) or a calcineurin inhibitor.

### Combination Treatment

In certain embodiments, the cell culture compositions may be administered to a subject in combination with a second agent. According to some embodiments, the second agent is a therapeutic agent.

The term “in combination with” also includes sequential or concomitant administration the cell culture composition and a second therapeutic agent. Administration “concurrent” or with the pharmaceutical composition comprising the cell culture composition described herein means that the additional therapeutic agent is administered to the subject in a separate dosage form within less than 5 minutes (before, after, or at the same time) of administration of the pharmaceutical composition comprising cell culture composition, or administered to the subject as a single combined dosage formulation comprising both the additional therapeutic agent and the cell culture composition.

In certain embodiments, the second therapeutic agent is a conventional therapeutic agent. A conventional therapeutic agent refers to therapeutic agents and drugs commonly or routinely used to treat AD in patients. Conventional therapeutic agents include systemic as well as topical therapeutics. For example, the most commonly or frequently prescribed drugs are the topical corticosteroids (TCS). Other examples of such agents include, but are not limited to, topical calcineurin inhibitors, anti-histamines, oral immunosuppressants, and glucocorticoids, systemic immunosuppressants such as methotrexate, cyclosporine, and azathioprine. Conventional therapeutic agents are used to relieve the symptoms of AD; however these agents have numerous and considerable adverse side effects including diabetes, hypertension, osteoporosis, myelosuppression, nephrotoxicity, hepatotoxicity, leucopenia, an increased risk of microbial infections. Topical agents such as corticosteroids and calcineurin inhibitors are not recommended for long-term application due to the risk of irreversible skin atrophy, dyspigmentation, acneiform eruptions and risks associated with systemic absorption including skin malignancies and lymphomas. Also repetitive application of any topical therapies over a long period of time can erode patient compliance.

In certain embodiments, the pharmaceutical composition comprising cell culture composition is administered to a subject in conjunction with a non-pharmaceutical therapy such as ultraviolet (UV) light therapy.

### Kits

The present disclosure also provides kits. In one aspect, a kit of the disclosure comprises (a) a composition of the disclosure and (b) instructions for use thereof. In another aspect, a kit of the disclosure comprises (a) any one of the live biotherapeutic compositions of the disclosure, and (b) instructions for use thereof. The compositions of the disclosure are described supra. In some embodiments, a composition of the disclosure is an engineered microorganism capable of expressing therapeutically relevant recombinant fusion polypeptides, as described supra.

The present disclosure is further illustrated by the following examples, which should not be construed as further limiting. The contents of all figures and all references, patents and published patent applications cited throughout this application, as well as the Figures, are expressly incorporated herein by reference in their entirety.

### EXAMPLES

The following examples further describe and demonstrate embodiments within the scope of the present disclosure. The Examples are given solely for purpose of illustration and are not to be construed as limitations of the present disclosure, as many variations thereof are possible without departing from the spirit and scope of the disclosure.

### Example 1. Plasmid Development

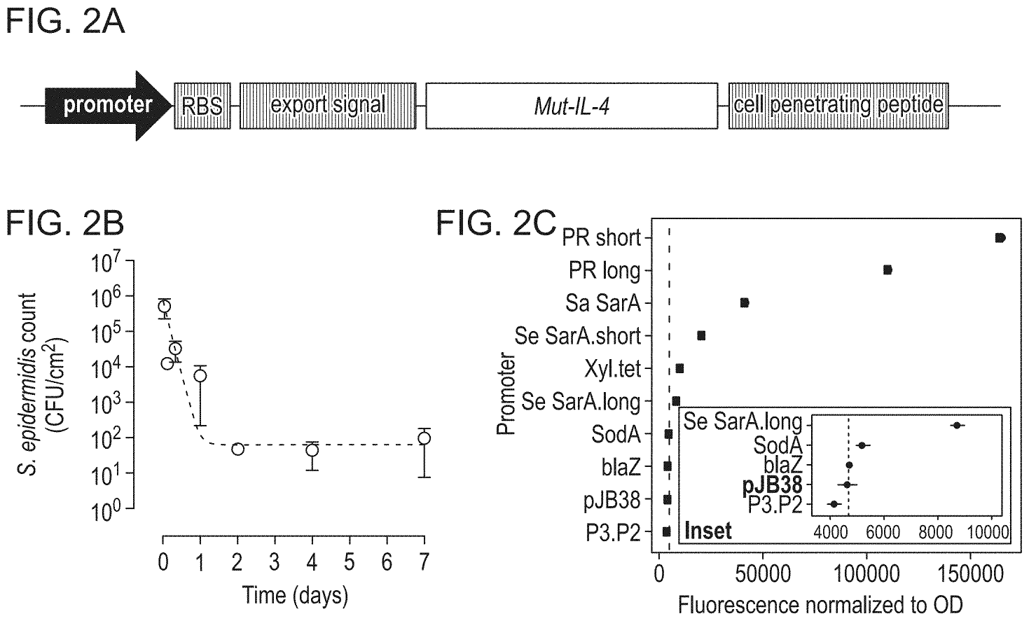

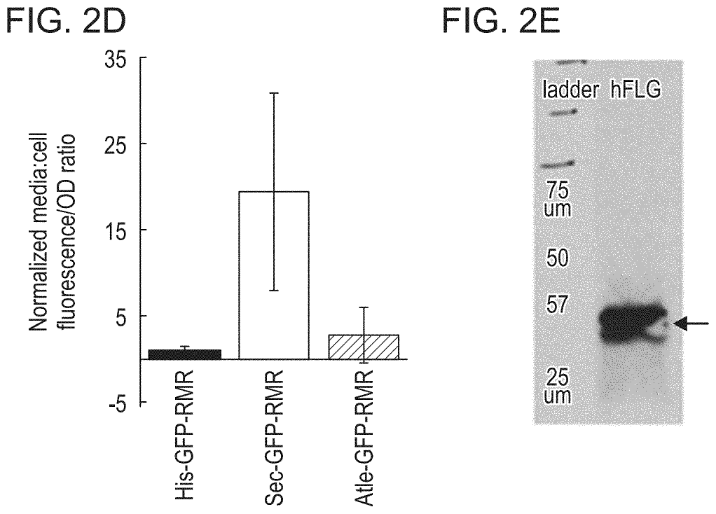

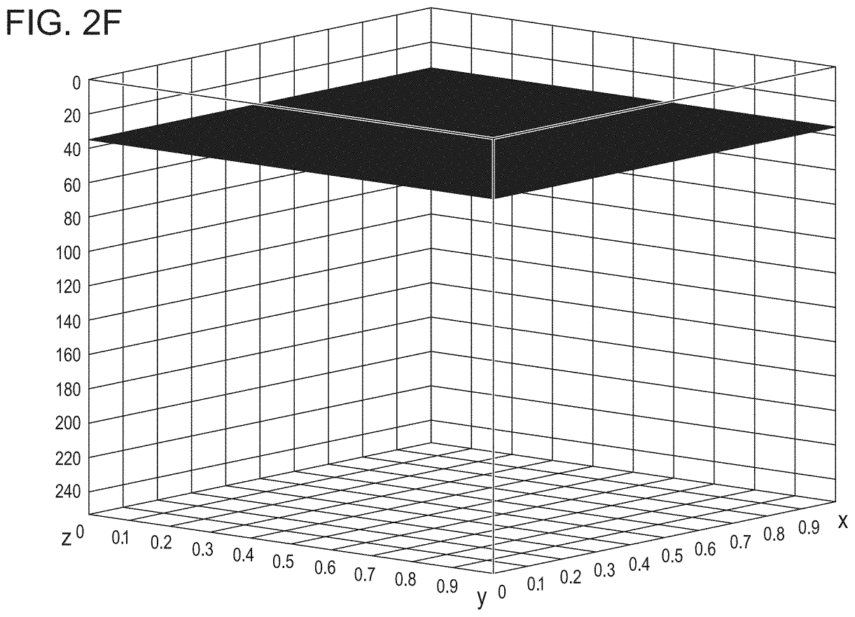

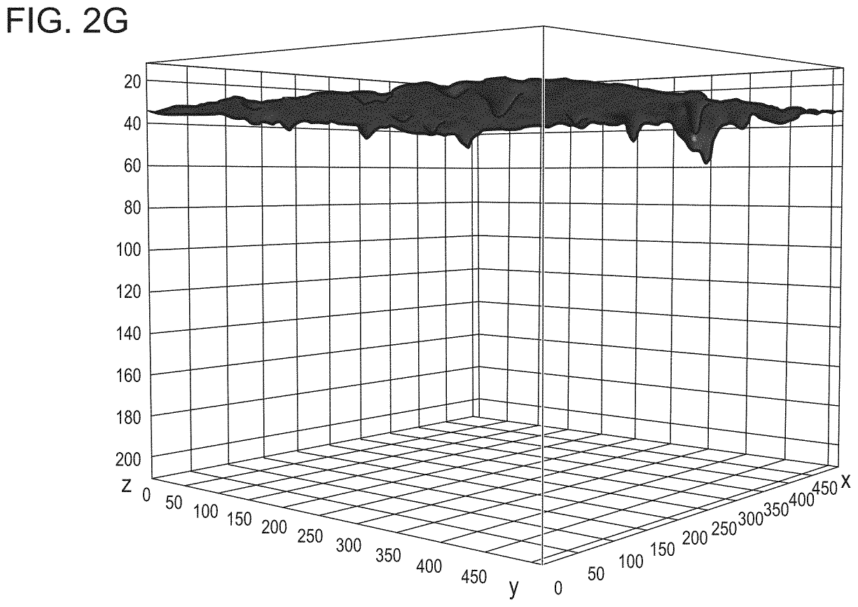

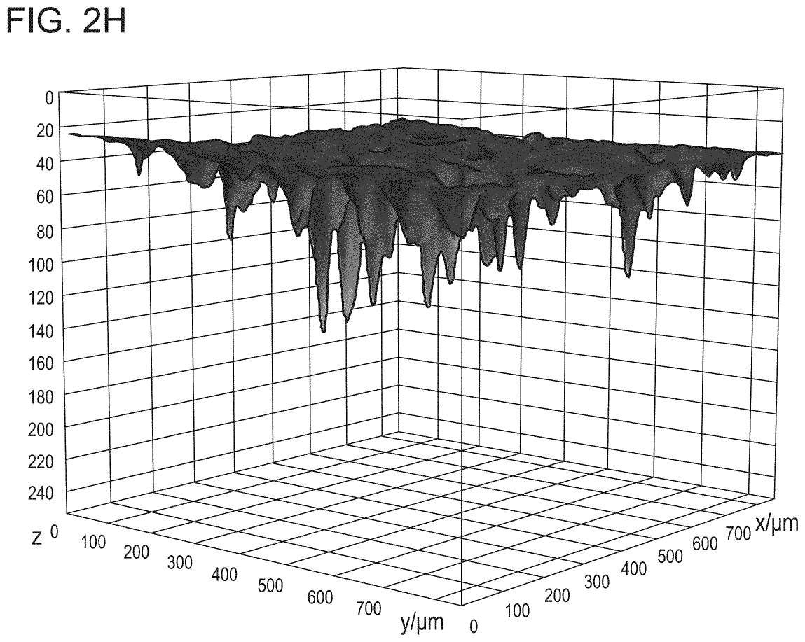

A general plasmid has been developed (FIG. 1) that can encode a protein that is exported out of the S. epidermidis cell and then imported into human keratinocytes. This plasmid, pAZT, is based on pJB38,⁴⁷ an allelic exchange E. coli-staphylococcal shuttle vector, where its functionality has been improved (FIG. 1 and FIG. 2A). The engineered S. epidermidis effectively colonizes reconstituted epidermis and is capable of producing up to 30-44 μg of protein per ˜108 CFU/mL and secreted protein in the media is approximately 1-2 μg/mL. Additional features, including colonization on mouse skin, optimization of promoter selection, evidence of a functional secretion signal, ability to produce human proteins, and ability to deliver protein through the stratum cornuem, are shown in FIG. 2B-I. Despite the formidable barrier properties of the epidermis, protein delivery through the stratum corneum has been demonstrated by employing transduction peptides or cell penetrating peptides (CPP). The construct utilizes an HIV trans-activator of a transcription-derived cell-penetration peptide (RMR) protein motif attached to the protein of interest, which allows trans-stratum corneum protein delivery (See FIG. 2F-I).

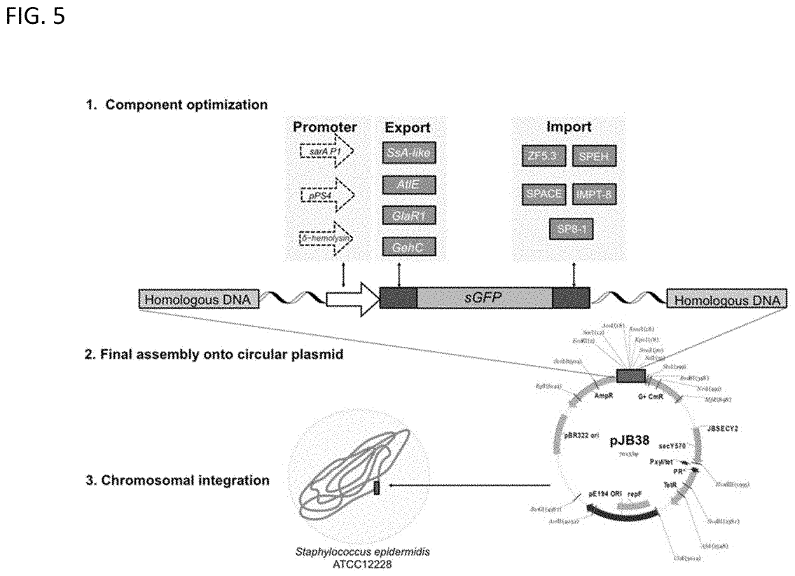

FIG. 5 is a schematic that shows exemplary recombinant constructs and integration in genome.

### Example 2. Characterization of the In Vitro Activity of [R121D, Y124D]-IL-4 Secreted by Staphylococcus epidermidis

The effective production and secretion of [R121D, Y124D]-IL-4 (mut-IL-4) will be demonstrated. A strain of Staphylococcus epidermidis (SE) has been designed that secretes this mutant IL-4. The activity of His-tag purified [R121D,Y124D]-IL-4 will be tested to determine in vitro inhibition of IL-4R and IL-13 using well-established assays.

### Purification of Recombinant Mut-IL-4

First, a source of purified mut-IL-4 protein will be generated to serve as an activity control for comparisons against mut-IL-4 purified or secreted from S. epidermidis. Recombinant E. coli strains that have cDNA fragments encoding mut-IL-4 in a BL21(DE3) host will be obtained. Production, purification, and verification of recombinant His-tagged IL-4 fragments will occur as previously described.²⁸

Purification of Recombinant IL-4 from S. epidermidis

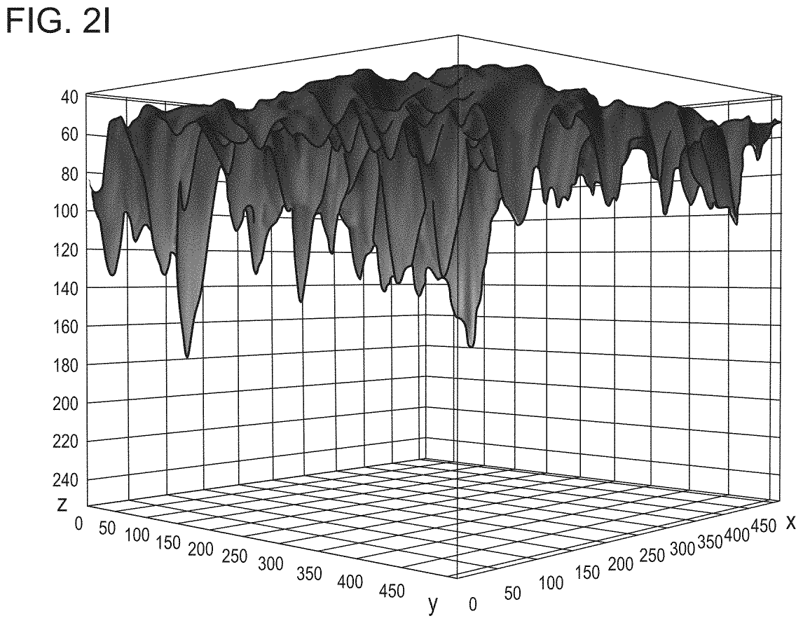

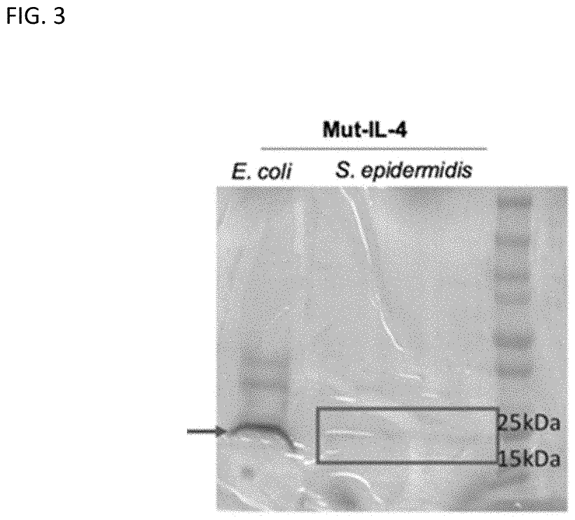

Next, it will be tested if recombinant mut-IL-4 produced by S. epidermidis has high activity in vitro. Using a similar design as in E. coli, a His-tagged version of [R121D, Y124D]-IL-4 (mut-IL-4) with a cleavable linker will be codon-optimized for expression in S. epidermidis. Standard protein purification methods will be used, and Western blot against mut-IL-4 using commercial antibodies (anti-IL-4) will be used to verify correct expression and purification. Results of mut-IL-4 expression in bacterial systems are presented in FIG. 3.

Exemplary cleavable peptide linkers that may be used include, but are not limited to:

(SEQ ID NO: 66)
-ENYFQG-
(SEQ ID NO: 67)
-IXGR-

where X is either D or E

(SEQ ID NO: 68)
-LVPRGSH-
(SEQ ID NO: 69)
-X1X2X3X4K-

where X₁, X₂, X₃ and X₄ are either D or E

(SEQ ID NO: 70)
-PGAAHY-
(SEQ ID NO: 71)
-LEVLFQG-

### Activity Characterization

Proliferation Assays

To characterize activity of mut-IL-4 in vitro, a suite of cell proliferation assays will be used to assess the effect of mut-IL-4 on facilitating or preventing proliferation of inflammatory cell types in vitro, using wild type IL-4 (commercially available) as a control. The cell proliferation assays that demonstrate activation under IL-4 or IL-13 include TF-1 cell proliferation (pre-leukemic human B cells, which express the shared IL13/4 receptor)^{53,54} and T cell proliferation assays,²⁶ which are commercially available and will be used in this study.

For each cell proliferation assay, 2,500 cells will be seeded/well in 100 μl of media in a 96-microtiter plate Immediately after seeding, the cells will be treated with wild type IL-4 or mutIL-4 alone or in combination at appropriate concentrations (0.1 ng/ml to 100 ng/ml). Proliferation will be measured between 60-70 hours post induction using the fluorometric cell proliferation assay kit (BioVision, CA Catalog #K307-1000).

### Biacore Binding Assay

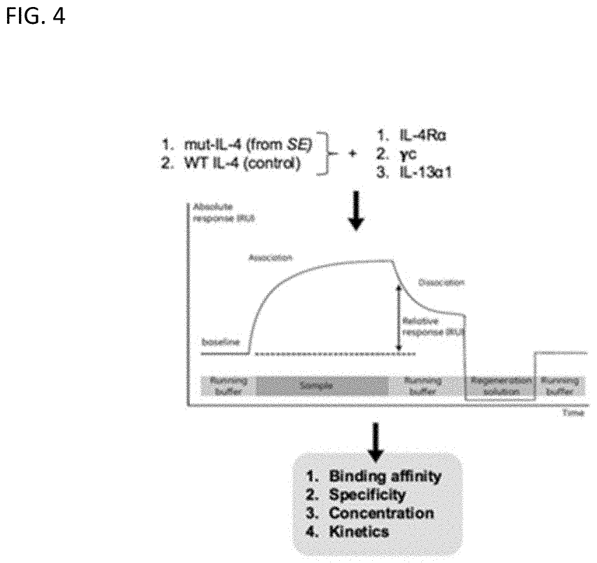

A keratin binding assay will be used to measure activity of mut-IL-4. This will be assessed with a Biacore (GE Healthcare, Zilina Region, Slovakia) surface plasmon resonance method to determine mut-IL-4 activity. This will measure the binding affinity, specificity, concentration, and kinetics to examine dynamics of binding between IL-4 or mut-IL-4 and IL-4Rα, γc, and IL-13α. Expected metrics produced by this method include Kₒₙ, K_{off}, K_{d}, and Kᵢ. The assay workflow is presented in FIG. 4.

The results from these experiments will demonstrate the in vitro activity of recombinant mut-IL-4 purified from and produced by S. epidermidis against IL-4/IL-13-driven responses in vitro to support the feasibility of mut-IL-4 heterologous production by S. epidermidis in vivo.

### Example 3. Evaluation of the In Vivo Activity of [R121D, Y124D]-IL-4-Secreting Staphylococcus epidermidis

Tape stripped mice exposed to antigen (which exhibit Th2-driven cutaneous inflammation) will be used. With these mice, application of topical [R121D, Y124D]-IL-4 as well as S. epidermidis secreting [R121D, Y124D]-IL-4 will be used to test for improved skin phenotypes to support the viability and efficacy of this therapeutic approach.

### Mouse Models

Skin barrier removal by tape stripping has been shown to polarize skin dendritic cells to promote a Th2 response upon allergen exposure.^{55,56} Damaging the skin by tape stripping increased skin levels of TSLP and the resulting Th2 response was dependent on TSLP signaling. Importantly, tape stripping and antigen exposure significantly results in elevated IL-4 and IL-13 production six hours after tape stripping,⁵⁵ which will be the key focus of these studies. A tape stripped mouse model (BALB/c background) as well as normal BALB/c mice (Jackson Laboratory, Bar Harbor, Me.) will be used. Tape stripping will be done according to published studies.⁵⁶ Briefly, one ear of BALB/c mice will be tape stripped 12-30 times with adhesive tape (Transpore surgical tape, 3M Health Care, St. Paul, Minn.). For each stripping, a fresh piece of tape will be lightly pressed onto the ear and pulled off. Then, OVA antigen will be applied to each ear. Mice will be sequestered for 12 hours before any treatment to allow for the appropriate immune response.

### Study Design

First, purified mouse [R121D, Y124D]-IL-4 (mut-IL-4) will be generated. Commercially available recombinant mouse wild type IL-4 will be used as controls. BALB/c mice will be tape stripped, and tape stripped and non-tape stripped mice will be randomized into the following treatment groups: topical vehicle control (50% glycerol, 50% sterilized TSA medium), topical recombinant wild-type mIL-4 (5 μg/cm² surface area), topical purified mut-IL-4 from SE (5 μg/cm² surface area), topical wild type SE (1.0×10⁸ CFU/cm² in 50% glycerol), and mut-IL-4-secreting SE constructs (SE^{mut-IL-4}) (10⁸ CFU/cm² in 50% glycerol). 12 hours after tape stripping, each solution will be applied to one ear every 12 hours for 48 hours, and mice will also be assessed at 48 hours, and the mice will be sacrificed according to animal protocols. The untreated ear for each mouse will serve as a control.

The primary outcomes that will be measured are change in IgE levels and pro-inflammatory cytokine levels (described below), which exploratory endpoints of exploring histological signs of inflammation, as well as phenotypic changes.

About 8 mice will be needed per arm per genotype in order to detect a mean change of 4 points in the clinical disease score between groups. This gives a total of 40 tape stripped BALB/c mice for the study.

### Measures of Inflammation

Variable expression of genes associated with AD development, progression, and maintenance (AD-associated pathogenic cytokines (IL-4, IL-5, IL-13, INF-γ, IL-17, IL-10 and TNF-α) will be measured by standard qPCR assays for both serum levels and cutaneous levels. Briefly, total RNA from both skin samples and total serum will be isolated using the Qiagen RNeasy Mini Kit (Qiagen, Valencia, Calif.) following the manufacturer's instructions. The respective cDNA will be synthesized using reverse transcriptase PCR (RT-PCR). Real-time PCR will be performed using the comparative 2-ΔΔCT method and will be normalized to Glyceraldehyde 3-phosphate dehydrogenase (GAPDH) transcript levels.

In addition, cells will be isolated to study other immunological changes. Keratinocytes, epidermal cells, and cells from the lymph nodes will be isolated. Single cell suspensions will be stained with either LIVE/DEAD Fixable Blue Dead Cell Stain Kit (Invitrogen) or 4′, 6-diamidino-2-phenylindol (DAPI, Sigma) in HBSS to exclude dead cells. For detection of transcription factors, cells will be stained using the Foxp3 staining set (eBioscience) according to the manufacturer's protocol. Cells will be stained with the following antibodies purchased from either eBioscience, BD Biosciences, or Dendritics Corp: CD4, IL-10, IL-17A, IFN-γ, TNF-α, Foxp3, CD34, CD44 and/or CD25 Staining will be performed in the presence of FcBlock (eBioscience), 0.2 mg/ml purified rat IgG and 1 mg/ml of normal mouse serum (Jackson Immunoresearch).

Histological and phenotypic changes in the skin, including spongiosis, angiosis, erethyma, etc., will be assessed.

Statistical Analyses

Differences between groups for the primary outcome, the macroscopic clinical disease score, will be assessed using two-sided student t-tests, if the data are normally distributed. Otherwise, nonparametric equivalents such as the Wilcoxon-rank sum test will be used. Differences across groups will be assessed with ANOVA or nonparametric equivalents (Kruskal-Wallis). Differences in ordinal variables will be assessed using Chi-square tests. All P-values will be corrected, if necessary, for multiple comparisons using false discovery rate.

Unless otherwise indicated, experiments will be performed in triplicates, and means and standard deviations will be reported. For comparisons between groups, two-sided t-test or analysis of variance will be used. If data are not normally distributed, these will be replaced with non-parametric equivalents (Wilcoxon-rank sum and Kruskal-Wallis tests).

Safety and “Kill Switches”

A key requirement for nearly all recombinant microorganisms for clinical use is the ability to prevent undesired introduction to other individuals or environments. In order to ensure safety of the engineered strain, the present invention, In some embodiments, uses an auxotrophic strain, which requires supplementation of key amino acids (D-ala) or a certain metabolic gene (AlaR) for survival, and simultaneously replaces the need for an antibiotic resistant strain for selection, the latter of which is not commercially viable. In another embodiment, the present invention integrates a “kill switch”, which is based on CRISPR/Cas9 self-cleavage upon induction of a dual xylose-riboswitch promoter. In yet another embodiment, the present invention provides cell counters, which recombine out the AZT gene expression cassette after a defined number of divisions, although this method would necessitate reapplication of the vehicle. To ensure the safety of the engineered S. epidermidis of the present invention, a CRISPR/Cas9-based kill switch, which is xylose-inducible and doubly regulated with a theophylline riboswitch, is used. The basis of this approach is that Cas9 is extremely efficient at chromosomal cleavage given a targeting guide, and since staphylococci lack canonical non-homologous end joining repair pathways, genomic cleavage results in death in the absence of a homologous recombination template. The use of a CRISPR-based system also confers great specificity, since comparative genomics can be used to design guides unique to the engineered S. epidermidis strain of the present invention, such that the construct is inactive if spread to other microbes by horizontal gene transfer. Finally, In some embodiments, the present invention provides a construct designed to express multiple CRISPR spacers to simultaneously target multiple genomic regions to ensure cleavage and minimize survival by reversion.

### EQUIVALENTS

Those skilled in the art will recognize or be able to ascertain using no more than routine experimentation, many equivalents to the specific embodiments of the disclosure described herein. Such equivalents are intended to be encompassed by the following claims.

### REFERENCES

1. Williams H, Robertson C, Stewart A, et al. Worldwide variations in the prevalence of symptoms of atopic eczema in the International Study of Asthma and Allergies in Childhood. J Allergy Clin Immunol. 1999; 103(1 Pt 1):125-138.
2. Shaw T E, Currie G P, Koudelka C W, Simpson E L. Eczema prevalence in the United States: data from the 2003 National Survey of Children's Health. J Invest Dermatol. 2011; 131(1):67-73.
3. Sullivan M, Silverberg N B. Current and emerging concepts in atopic dermatitis pathogenesis. Clinics in dermatology. 2017; 35(4):349-353.
4. Hoare C, Li Wan Po A, Williams H. Systematic review of treatments for atopic eczema. Health Technol Assess. 2000; 4(37):1-191.
5. Whiteley J, Emir B, Seitzman R, Makinson G. The Burden of Atopic Dermatitis in U.S. Adults: Results from the 2013 National Health and Wellness Survey. Current Medical Research and Opinion. 2016:1-32.
6. Nelms K, Keegan A D, Zamorano J, Ryan J J, Paul W E. The IL-4 receptor: signaling mechanisms and biologic functions. Annual review of immunology. 1999; 17:701-738.
7. Mueller T D, Zhang J-L, Sebald W, Duschl A. Structure, binding, and antagonists in the IL-4/IL-13 receptor system. Biochimica et Biophysica Acta (BBA)—Molecular Cell Research. 2002; 1592(3):237-250.
8. Hamid Q, Boguniewicz M, Leung D Y. Differential in situ cytokine gene expression in acute versus chronic atopic dermatitis. The Journal of clinical investigation. 1994; 94(2):870-876.
9. Sidbury R, Khorsand K. Evolving Concepts in Atopic Dermatitis. Current allergy and asthma reports. 2017; 17(7):42.
10. Howell M D, Kim B E, Gao P, et al. Cytokine modulation of atopic dermatitis filaggrin skin expression. The Journal of allergy and clinical immunology. 2009; 124(3 Suppl 2):R7-r12.
11. Kim B E, Leung D Y, Boguniewicz M, Howell M D. Loricrin and involucrin expression is down-regulated by Th2 cytokines through STAT-6. Clinical immunology (Orlando, Fla.). 2008; 126(3):332-337.
12. Hatano Y, Terashi H, Arakawa S, Katagiri K. Interleukin-4 suppresses the enhancement of ceramide synthesis and cutaneous permeability barrier functions induced by tumor necrosis factor-alpha and interferon-gamma in human epidermis. The Journal of investigative dermatology. 2005; 124(4):786-792.
13. Kobayashi J, Inai T, Morita K, et al. Reciprocal regulation of permeability through a cultured keratinocyte sheet by IFN-γ and IL-4. Cytokine. 2004; 28(4):186-189.
14. Shirakawa T, Deichmann K A, Izuhara K, Mao X-Q, Adra C N, Hopkin J M. Atopy and asthma: genetic variants of IL-4 and IL-13 signalling. Immunology Today. 21(2):60-64.
15. Stott B, Lavender P, Lehmann S, Pennino D, Durham S, Schmidt-Weber C B. Human IL-31 is induced by IL-4 and promotes TH2-driven inflammation. The Journal of allergy and clinical immunology. 2013; 132(2):446-454.e445.
16. Sonkoly E, Muller A, Lauerma A I, et al. IL-31: a new link between T cells and pruritus in atopic skin inflammation. The Journal of allergy and clinical immunology. 2006; 117(2):411-417.
17. Kong H H, Oh J, Deming C, et al. Temporal shifts in the skin microbiome associated with disease flares and treatment in children with atopic dermatitis. Genome research. 2012; 22(5):850-859.
18. Kobayashi T, Glatz M, Horiuchi K, et al. Dysbiosis and Staphylococcus aureus Colonization Drives Inflammation in Atopic Dermatitis. Immunity. 2015; 42(4):756-766.
19. Seite S, Flores G E, Henley J B, et al. Microbiome of affected and unaffected skin of patients with atopic dermatitis before and after emollient treatment. Journal of drugs in dermatology: JDD. 2014; 13(11):1365-1372.
20. Williams M R, Gallo R L. Evidence that Human Skin Microbiome Dysbiosis Promotes Atopic Dermatitis. The Journal of investigative dermatology. 2017; 137(12):2460-2461.
21. Nakatsuji T, Chen T H, Narala S, et al. Antimicrobials from human skin commensal bacteria protect against Staphylococcus aureus and are deficient in atopic dermatitis. Science translational medicine. 2017; 9(378).
22. Nomura I, Goleva E, Howell M D, et al. Cytokine milieu of atopic dermatitis, as compared to psoriasis, skin prevents induction of innate immune response genes. Journal of immunology (Baltimore, Md.: 1950). 2003; 171(6):3262-3269.
23. Ong P Y, Ohtake T, Brandt C, et al. Endogenous antimicrobial peptides and skin infections in atopic dermatitis. The New England journal of medicine. 2002; 347(15):1151-1160.
24. Simpson E L, Gadkari A, Worm M, et al. Dupilumab therapy provides clinically meaningful improvement in patient-reported outcomes (PROs): A phase randomized, placebo-controlled, clinical trial in adult patients with moderate to severe atopic dermatitis (A D). Journal of the American Academy of Dermatology. 2016; 75(3):506-515.
25. Kruse N, Shen B J, Arnold S, Tony H P, Muller T, Sebald W. Two distinct functional sites of human interleukin 4 are identified by variants impaired in either receptor binding or receptor activation. The EMBO journal. 1993; 12(13):5121-5129.
26. An interleukin 4 (IL-4) mutant protein inhibits both IL-4 or IL-13-induced human immunoglobulin G4 (IgG4) and IgE synthesis and B cell proliferation: support for a common component shared by IL-4 and IL-13 receptors. The Journal of Experimental Medicine. 1993; 178(6):2213-2218.
27. Tomkinson A, Duez C, Cieslewicz G, et al. A Murine IL-4 Receptor Antagonist That Inhibits IL-4- and IL-13-Induced Responses Prevents Antigen-Induced Airway Eosinophilia and Airway Hyperresponsiveness. The Journal of Immunology. 2001; 166(9):5792-5800.
28. Kruse N, Lehrnbecher T, Sebald W. Site-directed mutagenesis reveals the importance of disulfide bridges and aromatic residues for structure and proliferative activity of human interleukin-4. FEBS letters. 1991; 286(1-2):58-60.
29. Belkaid Y, Segre J A. Dialogue between skin microbiota and immunity. Science. 2014; 346(6212):954-959.
30. Belkaid Y, Tamoutounour S. The influence of skin microorganisms on cutaneous immunity. Nature reviews Immunology. 2016; 16(6):353-366.
31. Structure, function and diversity of the healthy human microbiome. Nature. 2012; 486(7402):207-214.
32. Wikoff W R, Anfora A T, Liu J, et al. Metabolomics analysis reveals large effects of gut microflora on mammalian blood metabolites. Proceedings of the National Academy of Sciences of the United States of America. 2009; 106(10):3698-3703.
33. Salzman N H, Hung K, Haribhai D, et al. Enteric defensins are essential regulators of intestinal microbial ecology. Nat Immunol. 2010; 11(1):76-82.
34. Kau A L, Ahern P P, Griffin N W, Goodman A L, Gordon J I. Human nutrition, the gut microbiome and the immune system. Nature. 2011; 474(7351):327-336.
35. Ravel J, Gajer P, Abdo Z, et al. Vaginal microbiome of reproductive-age women. Proceedings of the National Academy of Sciences of the United States of America. 2011; 108 Suppl 1:4680-4687.
36. Grice E A, Segre J A. The skin microbiome. Nature reviews Microbiology. 2011; 9(4):244-253.
37. Diaz Heijtz R, Wang S, Anuar F, et al. Normal gut microbiota modulates brain development and behavior. Proceedings of the National Academy of Sciences of the United States of America. 2011; 108(7):3047-3052.
38. Weyrich L S, Dixit S, Farrer A G, Cooper A J, Cooper A J. The skin microbiome: Associations between altered microbial communities and disease. The Australasian journal of dermatology. 2015.
39. Oh J, Byrd A L, Deming C, Conlan S, Kong H H, Segre J A. Biogeography and individuality shape function in the human skin metagenome. Nature. 2014; 514(7520):59-64.
40. Oh J, Freeman A F, Park M, et al. The altered landscape of the human skin microbiome in patients with primary immunodeficiencies. Genome research. 2013; 23 (12):2103-2114.
41. Grice E A. The skin microbiome: potential for novel diagnostic and therapeutic approaches to cutaneous disease. Seminars in cutaneous medicine and surgery. 2014; 33(2):98-103.
42. Powers C E, McShane D B, Gilligan P H, Burkhart C N, Morrell D S. Microbiome and pediatric atopic dermatitis. The Journal of dermatology. 2015.
43. Cogen A L, Yamasaki K, Sanchez K M, et al. Selective antimicrobial action is provided by phenol-soluble modulins derived from Staphylococcus epidermidis, a normal resident of the skin. J Invest Dermatol. 2010; 130(1):192-200.
44. Cogen A L, Yamasaki K, Muto J, et al. Staphylococcus epidermidis antimicrobial delta-toxin (phenol-soluble modulin-gamma) cooperates with host antimicrobial peptides to kill group A Streptococcus. PloS one. 2010; 5(1):e8557.
45. Monk I R, Shah I M, Xu M, Tan M W, Foster T J. Transforming the untransformable: application of direct transformation to manipulate genetically Staphylococcus aureus and Staphylococcus epidermidis. mBio. 2012; 3(2).
46. Wei W, Cao Z, Zhu Y L, et al. Conserved genes in a path from commensalism to pathogenicity: comparative phylogenetic profiles of Staphylococcus epidermidis RP62A and ATCC12228. BMC genomics. 2006; 7:112.
47. Bose J L, Fey P D, Bayles K W. Genetic tools to enhance the study of gene function and regulation in Staphylococcus aureus. Applied and environmental microbiology. 2013; 79(7):2218-2224.
48. Nodake Y, Matsumoto S, Miura R, et al. Pilot study on novel skin care method by augmentation with Staphylococcus epidermidis, an autologous skin microbe—A blinded randomized clinical trial. Journal of dermatological science. 2015; 79(2):119-126.
49. Naik S, Bouladoux N, Linehan J L, et al. Commensal-dendritic-cell interaction specifies a unique protective skin immune signature. Nature. 2015; 520(7545):104-108.
50. Li D, Lei H, Li Z, Li H, Wang Y, Lai Y. A novel lipopeptide from skin commensal activates TLR2/CD36-p38 MAPK signaling to increase antibacterial defense against bacterial infection. PloS one. 2013; 8(3):e58288.
51. Lai Y, Cogen A L, Radek K A, et al. Activation of TLR2 by a small molecule produced by Staphylococcus epidermidis increases antimicrobial defense against bacterial skin infections. J Invest Dermatol. 2010; 130(9):2211-2221.
52. Naik S, Bouladoux N, Wilhelm C, et al. Compartmentalized control of skin immunity by resident commensals. Science. 2012; 337(6098):1115-1119.
53. McKenzie A N, Culpepper J A, de Waal Malefyt R, et al. Interleukin 13, a T-cell-derived cytokine that regulates human monocyte and B-cell function. Proceedings of the National Academy of Sciences of the United States of America. 1993; 90(8):3735-3739.
54. Thompson J P, Debinski W. Mutants of Interleukin 13 with Altered Reactivity toward Interleukin 13 Receptors. Journal of Biological Chemistry. 1999; 274(42):29944-29950.
55. Oyoshi M K, Larson R P, Ziegler S F, Geha R S. Mechanical injury polarizes skin dendritic cells to elicit a T(H)2 response by inducing cutaneous thymic stromal lymphopoietin expression. The Journal of allergy and clinical immunology. 2010; 126(5):976-984, 984.e971-975.
56. Holzmann S, Tripp C H, Schmuth M, et al. A Model System Using Tape Stripping for Characterization of Langerhans Cell-Precursors In Vivo. Journal of Investigative Dermatology. 2004; 122(5): 1165-1174.

1. A recombinant microorganism capable of secreting a polypeptide, wherein the recombinant microorganism comprises an expression vector comprising a first coding sequence comprising a gene capable of expressing the polypeptide;
and a second coding sequence comprising a gene capable of expressing a cell penetrating peptide.

2. The recombinant microorganism of claim 1, further comprising a third coding sequence comprising a gene capable of expressing an export or secretion or signal peptide.

3. The recombinant microorganism of claim 1, further comprising a third or fourth coding sequence comprising a pro-peptide.

4. The recombinant microorganism of claim 1, wherein the expression of the first coding sequence, second coding sequence, third coding sequence and/or fourth coding sequence is under the control of one or more promoters.

5. (canceled)

6. (canceled)

7. (canceled)

8. The recombinant microorganism of claim 1, wherein the polypeptide is a mutant IL-4, or a variant thereof.

9. (canceled)

10. (canceled)

11. The recombinant microorganism of claim 8, wherein the mutant IL-4 is [R121D,Y124D]-IL-4.

12. The recombinant microorganism of claim 1, wherein the microorganism is selected from the group consisting of Bifidobacterium, Brevibacterium, Cutibacterium (formerly known as Propionibacterium), Lactococcus, Streptococcus, Staphylococcus, Lactobacillus, Enterococcus, Pediococcus, Leuconostoc, and Oenococcus, or combinations thereof.

13. (canceled)

14. A method for producing a live biotherapeutic composition, the method comprising:
(a) transfecting a cell with (i) a first coding sequence comprising a nucleic acid sequence capable of expressing a therapeutic polypeptide, and (ii) a second coding sequence comprising a nucleic acid sequence capable of expressing a cell penetrating peptide; and
(b) allowing the transfected cell to produce a therapeutic polypeptide fusion protein; and
(c) obtaining the live biotherapeutic composition.

15. The method of claim 14, further comprising (iii) transfecting the cell with a third coding sequence comprising a nucleic acid sequence capable of expressing an export or secretion or signal peptide and/or a fourth coding sequence comprising a nucleic acid sequence capable of expressing a pro-peptide.

16. The method of claim 15, wherein the first coding sequence, second coding sequence, third coding sequence and/or fourth coding sequence are arranged in a single plasmid.

17. The method of claim 15, wherein the arrangement of the first coding sequence, second coding sequence, third coding sequence and/or fourth coding sequence are operably linked to one or more promoters.

18. The method of claim 14, wherein the cell is selected from the group consisting of wherein the microorganism is selected from the group consisting of Bifidobacterium, Brevibacterium, Cutibacterium (formerly known as Propionibacterium), Lactococcus, Streptococcus, Staphylococcus, Lactobacillus, Enterococcus, Pediococcus, Leuconostoc, or Oenococcus, or combinations thereof.

19. (canceled)

20. The method of claim 14, wherein the therapeutic polypeptide is a mutant IL-4, or a variant thereof.

21. (canceled)

22. (canceled)

23. A composition obtained by the method of claim 14.

24. (canceled)

25. A live biotherapeutic composition comprising a recombinant microorganism wherein the recombinant microorganism comprises
(i) a first coding sequence comprising a nucleic acid sequence encoding a therapeutic polypeptide;
(ii) a second coding sequence comprising a nucleic acid sequence encoding a cell penetrating peptide;
(iii) a third coding sequence comprising a nucleic acid sequence encoding an export or secretion or signal peptide; and/or
(iv) a fourth coding sequence comprising a nucleic acid sequence encoding a pro-peptide; and
(v) a promoter operably linked to the first coding sequence, the second coding sequence, the third coding sequence and/or the fourth coding sequence;
wherein the recombinant microorganism comprising the first coding sequence, second coding sequence, third coding sequence and/or fourth coding sequence, is capable of expressing an IL-4 mutant polypeptide, or variant thereof.

26. (canceled)

27. (canceled)

28. (canceled)

29. (canceled)

30. (canceled)

31. A method of treating an abnormal skin condition comprising administering to a subject in need thereof the composition of claim 25.

32. The method according to claim 31, wherein the cell culture composition is a living cell culture composition.

33. The method according to claim 31, wherein the skin condition is atopic dermatitis (AD).

34. (canceled)

35. (canceled)

36. (canceled)

37. (canceled)

38. (canceled)

39. A mutant IL-4 polypeptide comprising [R121D, Y124D]-IL-4.

40. The mutant IL-4 polypeptide of claim 39, wherein the polypeptide is codon optimized.
